(19) United States
(12) Patent Application Publication
(10) Pub. No.: US 2012/0215075 A1
(43) Pub. Date: Aug. 23, 2012

(54) CORPSMAN/MEDIC MEDICAL ASSISTANT SYSTEM AND METHOD

(75) Inventors: Lawrence J. SURACE, Skaneateles, NY (US); Victor J. Riley, Hubert, NC (US); Kevin R. Lefebvre, Manlius, NY (US); Daniel R. Sommers, Skaneateles, NY (US); Michael E. Borden, JR., Camillus, NY (US); Geoffrey Lloyd, Jamesville, NY (US); Robert B. White, Jamesville, NY (US); Jeremy F. Belge, Baldwinsville, NY (US); Gregory W. Bintz, Clay, NY (US); Thomas V. Michlovitch, Cicero, NY (US); Michael G. Skrzypek, Cicero, NY (US)

(73) Assignee: SAAB SENSIS CORPORATION, East Syracuse, NY (US)

(21) Appl. No.: 13/297,665

(22) Filed: Nov. 16, 2011

Related U.S. Application Data
(63) Continuation of application No. PCT/US2010/035547, filed on May 20, 2010.
(60) Provisional application No. 61/179,948, filed on May 20, 2009.

(51) Int. Cl. A61B 5/00

(52) U.S. Cl. 600/301

(57) ABSTRACT

The present invention is a corpsman/medic medical assistant system that includes a portable transmit/receive pack, sensor leads for first and second physiological sensor subsystems and a corpsman/medic medical assistant control unit that includes a wireless transceiver. The portable transmit/receive pack has a sealed housing that includes the first and second physiological sensor subsystems, at least one processor running at least one algorithm, a wireless transceiver, and memory with a front panel that includes a display, at least one event key, a treatment button for recording treatment data, a timer, an observation button for recording observation data, and a plurality of physiological trend indicators. The algorithm determines a current physiological trend status of the patient based on the plurality of physiological measurements, and the transmit/receive pack links to the control unit by the wireless transceiver and receives the current physiological trend status of the patient.

### FIELD OF INVENTION

The present invention relates to portable medical monitoring devices for monitoring and recording a plurality of patient physiological parameters during level I, II and III care. The present invention provides a record of the plurality of patient physiological parameters of the patient, care giver observations, treatments administered and provides a printout of the recorded data in tabular and graphic form.

### BACKGROUND OF THE INVENTION

During emergency situations, such as vehicle accidents, natural disasters or battlefield situations, the first responder/on-scene care giver (i.e., level I care) is primarily focused on the stabilization of a patient for transport to a medical care facility environment (e.g., regional hospital, field hospital). During these emergency situations, very little if any data is electronically recorded and retained to provide a history of treatment for use at the medical care facility in diagnosis and treatment of the patient. In most emergency situations, the first responder has minimal face-to-face contact with the medical care providers at the hospital and thus is forced to provide summarized data that could miss information critical to the treatment of the patient.

In some rare cases, an emergency data card may be filled out by the first responder and used as a written record to highlight certain treatment information, such as medications administered and time administered to the patient at the scene of the accident and possibly observations by the first responder, but patient vital sign history data is not recorded and thus not available to the downstream care provider.

In situations were an emergency data card is used, when the patient is transferred to the care of emergency personnel (i.e., level II care), such as emergency medical technicians (EMTs) in ambulances, or helicopter/aircraft airlift (mercy flights), for transport to a primary care facility, the EMT may supplement the data that was transferred from the first responder to the EMT. For example, during transit the EMT may record additional medications administered and EMT observations on the emergency data card, or may use an additional data card to record such information. Patient vital sign data may be monitored during transit but is typically not recorded or transmitted to the medical care facility (level III care) personnel.

Due to the lack of consistent recording of such data, when patient hand-off to the medical care facility (i.e., level III care), such as a hospital, is completed, the EMT transfers the patient to the medical care facility personnel with, in almost all cases, incomplete vital sign recorded data and correlation to medications administered and observation data. The lack of a more complete record for the patient impedes the medical care facility personnel's ability to quickly provide the necessary care for critically injured patients. Further, patients may be covered with dirt, blood and other materials related to their injuries, and personnel receiving the patient may remove soiled/bloody clothing upon arrival, which may cause an emergency data card attached to the clothing to be misplaced or even discarded.

What is needed is a system and method for automatic collection, recording and presentation of pre-hospital patient vital sign data, as well as treatments administered, such as medications, and observations from both the on-site first responder (level I), emergency transport personnel (level II) and initial triage at the medical care facility (level III) to assist personnel at the medical care facility in the diagnosis and treatment of the patient and to prevent potential over-administering of medications during subsequent care procedures (e.g., anesthesia during operations).

### SUMMARY OF THE INVENTION

A corpsman/medic medical assistant system comprising a corpsman/medic medical assistant portable transmit/receive pack comprising a sealed housing that includes a first physiological sensor subsystem, a second physiological sensor subsystem, at least one processor running at least one algorithm, a power supply, a wireless transceiver, and memory, the sealed housing having a front panel that includes a display, at least one event keys, a treatment button for recording treatment data for treatments administered to a patient by a user, a timer, an observation button for recording observation data by the user, and a plurality of physiological trend indicators, sensor leads for the first physiological sensor subsystem and the second physiological sensor subsystem, and a corpsman/medic medical assistant control unit comprising an wireless transceiver. The display displays a plurality of physiological measurements for the patient when the sensor leads for the first physiological sensor subsystem and the second physiological sensor subsystem are connected between the patient and the transmit/receive pack, and the algorithm determines a current physiological trend status of the patient based on the plurality of physiological measurements from the first physiological sensor subsystem and the second physiological sensor subsystem and displays the current physiological trend status of the patient by activating one of the plurality of physiological trend indicators on the front panel. The transmit/receive pack is linked to the corpsman/medic medical assistant control unit by the wireless transceiver and the corpsman/medic medical assistant control unit receives the current physiological trend status of the patient from the transmit/receive pack. In one embodiment, the corpsman/medic medical assistant control unit transmits treatment data and observation data to the transmit/receive pack and the transmit/receive pack time stamps and stores the received treatment data and observation data in memory.

In one embodiment of the present invention, the timer in the transmit/receive pack is an elapsed time counter counting time from the initialization of the transmit/receive pack. In this embodiment, recorded time from the timer is converted to real-time when the transmit/receive pack is connected to an external PC. In another embodiment, the timer is a real-time clock that counts real-time from the initialization of the transmit/receive pack. The transmit/receive pack time stamps the plurality of physiological measurements for the patient using the timer, timestamps treatment data entered via the treatment button and observation data entered via the observation button on the front panel, and stores the plurality of physiological measurements for the patient, the treatment data and the observation data in memory.

In one embodiment, the corpsman/medic medical assistant control unit is a smart phone, PDA, tablet PC or other wireless device capable of connecting and displaying vital sign measurements from at least the first physiological sensor subsystem and the second physiological sensor subsystem. In this embodiment, the corpsman/medic medical assistant control unit displays current physiological trend status for a plurality of patients on one screen. When the current physiological status of the patient changes, the transmit/receive pack displays the new physiological status of the patient by activating another of the plurality of physiological trend indicators in a blinking pattern while the one physiological trend indicator is still activated in a solid pattern for a predefined period of time.

In one embodiment, the algorithm in the transmit/receive pack compares at least one of the current physiological measurements and historical trend data of the patient with threshold physiological values for each of the plurality of physiological measurements to determine the current physiological status of the patient. In another embodiment, the transmit/receive pack records a continuous waveform for at least one of the first physiological sensor subsystem and the second physiological sensor subsystem.

In one embodiment, the transmit/receive pack further comprises at least one external interface connector. In this embodiment, at least one other external physiological sensor can be connected to the transmit/receive pack via the external interface connector and the transmit/receive pack stores data received from the other external physiological sensors in memory. In another embodiment, the algorithm uses the data from the other external physiological sensors when determining the current physiological status of the patient.

In one embodiment, the portable transmit/receive pack continuously determines the status of the patient when a fault condition exists for one of first physiological sensor subsystem and the second physiological sensor subsystem. In another embodiment, the transmit/receive pack timestamps and records all faults from the first and second physiological sensor subsystems in memory.

In one embodiment, when the transmit/receive pack is connected to an external PC, the transmit/receive pack automatically formats the time stamped data recorded in memory including the physiological measurements of the patient over time, the treatment data, the observation data and sensor fault data into a spreadsheet format and a graphical format for printing.

In one embodiment, the transmit/receive pack further comprises a pneumatic exhaust vent having exhaust vent fins that protect against clogging of the pneumatic exhaust vent. In another embodiment, the wireless transceiver is a Bluetooth transceiver. In one embodiment, the transmit/receive pack further comprises an operational mode switch, wherein the display, treatment and observation buttons and plurality of physiological trend indicators on the front panel are turned off by positioning the switch to a “Silent” mode. In another embodiment, the transmit/receive pack further comprises means for attaching the transmit/receive pack to the patient.

In one embodiment, the transmit/receive pack is at least partially powered by an external power source through the at least one external connector. In another embodiment, one of the transmit/receive packs is powered by another transmit/receive pack to retrieve and store data recorded on said one of the transmit/receive packs.

In another embodiment, the portable transmit receive pack comprises a sealed housing that includes a first physiological sensor subsystem, a second physiological sensor subsystem, at least one processor running at least one algorithm, a power supply, and memory, the sealed housing having a front panel that includes a display, at least one event key, a treatment button for recording treatment data for treatments administered to a patient by a user, a timer, an observation button for recording observation data by the user and a plurality of physiological trend indicators and sensor leads for the first physiological sensor subsystem and at least the second physiological sensor subsystem. The display displays a plurality of physiological measurements for the patient when the sensor leads for the first physiological sensor subsystem and the second physiological sensor subsystem are connected between the patient and the transmit/receive pack, and the algorithm determines a current physiological trend status of the patient based on the plurality of physiological measurements from the first physiological sensor subsystem and the second physiological sensor subsystem and displays the current physiological trend status of the patient by activating one of the plurality of physiological trend indicators on the front panel.

In one embodiment, the transmit/receive pack time stamps the plurality of physiological measurements for the patient using the timer and stores the plurality of physiological measurements for the patient in memory. In another embodiment, the transmit/receive pack timestamps treatment data entered via the treatment button and observation data entered via the observation button on the front panel of the transmit/receive pack and stores the treatment data and observation data in memory. In another embodiment, the transmit/receive pack receives treatment data and observation data from the corpsman/medic medical assistant control unit and the transmit/receive pack time stamps and stores the received treatment data and observation data in memory.

In one embodiment, the transmit/receive pack further comprises a wireless transceiver, wherein the transmit/receive pack is linked to a corpsman/medic medical assistant control unit by the wireless transceiver, the wireless transceiver transmits the current physiological status of the patient to the corpsman/medic medical assistant control unit and the corpsman/medic medical assistant control unit displays the current physiological status of the patient to the user. In another embodiment, the transmit/receive pack further comprises at least one external interface connector, wherein other external physiological sensors are connected to the transmit/receive pack via the at least one external interface connector and the transmit/receive pack stores data received from other external physiological sensors in memory. In yet another embodiment, the algorithm uses the data from the other external physiological sensors when determining the current physiological status of the patient.

In one embodiment, the algorithm compares the current physiological measurements of the patient with predetermined physiological values for each of the plurality of physiological measurements to determine the current physiological status of the patient and displays the current physiological status of the patient by activating one of the plurality of physiological trend indicators. In one embodiment, when the current physiological status of the patient changes, the transmit/receive pack displays the new physiological status of the patient by activating another of the plurality of physiological trend indicators in a blinking pattern while the one physiological trend indicator is still activated in a solid pattern for a predefined period of time.

In one embodiment, the transmit/receive pack timestamps and records all faults from the first physiological sensor subsystem and the second physiological sensor subsystem in memory. In one embodiment, the transmit/receive pack records a continuous waveform for at least one of the first physiological sensor subsystem and the second physiological sensor subsystem. In one embodiment, the transmit/receive pack further comprises means for attaching the transmit/receive pack to the patient.

In one embodiment, when the transmit/receive pack is connected to an external PC, the transmit/receive pack automatically formats the time stamped data recorded in memory including the physiological measurements of the patient over time, treatment data, observation data and sensor fault data into a spreadsheet format and a graphical format for printing. In another embodiment, the transmit/receive pack further comprises a pneumatic exhaust vent having exhaust vent fins that protect against clogging of the pneumatic exhaust vent.

In one embodiment, the timing source is an elapsed time counter and the elapsed time counter counts time from the initialization of the transmit/receive pack. In this embodiment, the recorded time from the timer is converted to real-time when the transmit/receive pack is connected to an external PC. In another embodiment, the timer is a real-time clock that counts real-time from the initialization of the transmit/receive pack.

In one embodiment, the wireless transceiver is a Bluetooth transceiver. In another embodiment, the transmit/receive pack further comprises at least one of a temperature sensor, a humidity sensor and an atmospheric pressure sensor, wherein the transmit/receive pack timestamps and records data received from said at least one sensor in memory. In yet another embodiment, the transmit/receive pack further comprises an operational mode switch, wherein the display, treatment and observation buttons and plurality of physiological trend indicators on the front panel of the transmit/receive pack are turned off by activating the operational mode switch to the “Silent” mode.

In one embodiment, the power supply is a rechargeable battery. In another embodiment, the transmit/receive pack is powered at least partially by an external power source through the at least one external connector. In another embodiment, a first transmit/receive pack is powered by a second transmit/receive pack to retrieve and store data recorded on the first transmit/receive pack. In yet another embodiment, the transmit/receive pack retains sequential time in memory for at least 72 hours after the power source is drained and does not support operation of the transmit/receive pack. In one embodiment, the transmit/receive pack retains data stored in memory for at least 30 days after the power source is drained and does not support operation of the transmit/receive pack.

In one embodiment, the transmit/receive pack further comprises a sealed package enclosing the transmit/receive pack, wherein the transmit/receive pack automatically initializes when removed from the sealed package. In this embodiment, the transmit/receive pack performs an operational system status check in the sealed package without compromising the sealed package.

### BRIEF DESCRIPTION OF THE DRAWINGS

FIG. 1 depicts one embodiment of the portable transmit/receive pack (PTRP) of the Corpsman/Medic Medical Assistant (CMMA) system according to the present invention;

FIG. 2 depicts the components of one embodiment of the integrated CMMA System;

FIGS. 3(a) and (b) show front and perspective views of one embodiment of the PTRP;

FIG. 4 is a block diagram of the internal components of one embodiment of the PTRP;

FIG. 5 shows the exhaust vent on one embodiment of the PTRP;

FIG. 6 shows the first embodiment of the PTRP connected to a patient;

FIG. 7 shows a State Diagram for one embodiment of the PTRP;

FIG. 8 shows the System Initialization display for one embodiment of the PTRP;

FIG. 9 shows one embodiment of the PTRP Front Panel Display during operation;

FIG. 10 shows the Trend Status Indicator Transition in one embodiment of the PTRP;

FIG. 11 shows the 120 V and 12 VDC charging configurations for one embodiment of the PTRP;

FIG. 12 shows a Single Sensor Fault display in one embodiment of the PTRP;

FIG. 13 shows a Double Sensor Fault display in one embodiment of the PTRP;

FIG. 14 shows an example of PTRP Downloaded Data in Spreadsheet format for one embodiment of the PTRP;

FIG. 15 shows an example of one embodiment of a Patient Casualty Card;

FIGS. 16(a) and (b) show examples of PTRP Downloaded Data in Graphical Format;

FIG. 17 shows one embodiment of an Integrated CMMA System;

FIG. 18 shows the Patient Tracking Screen in one embodiment of the CMMACU;

FIG. 19 shows the Observations Display Screen in one embodiment of the CMMACU;

FIG. 20 shows the Medications Display Screen in one embodiment of the CMMACU;

FIG. 21 shows the Personal Data Entry Display Screen in one embodiment of the CMMACU;

FIG. 22 shows the internal components within the PTRP in the first example of the PTRP;

FIG. 23 shows an Algorithm Block Diagram for the first example of the PTRP;

FIG. 24 shows the Front Panel of the first example of the PTRP;

FIG. 25 shows the Treatment Display Confirmation Screen of the first example of the PTRP; and

FIG. 26 shows the Observation Display Confirmation Screen of the first example of the PTRP.

### DETAILED DESCRIPTION OF THE INVENTION

The Corpsman/Medic Medical Assistant (CMMA) is a lightweight, portable, easy to use vital sign data collection, data recording, and data fusion system. The PTRP automatically collects vital sign data for an injured party (e.g., patient), as well as any treatment data and observation information from first responder and emergency transport personnel (e.g., EMTs) before the patient arrives at the medical care facility. The PTRP automatically stores the patient vital sign data and, when connected to a personal computer without any function specific software, will provide a printable record of the patient's vital sign data, treatment data and observation information in both a tabular format and a graphical format.

The CMMA system provides a significant improvement in patient care by capturing, storing and reporting vital sign data for patients throughout all phases of the continuum of care. The CMMA provide useful information to the first responder from the moment of attachment at the initial incident or point of injury and continues to be a viable recorder of vital signs, treatments and observations during initial treatment and transport to a medical facility and builds a composite medical history/care report at the medical facility. Since the primary focus of the CMMA is to notify and report multiple levels of patient physiological status, the CMMA assumes that the medical condition of the patient is a direct result of an emergency situation or a traumatic event. Automatically collecting this information and presenting it to hospital personnel in a familiar and easily interpreted format is critical to improving the survival rate of patients, particularly when they have received blunt force trauma injuries, such as vehicle accidents and explosion blasts.

In the initial phase of care from the first provider (i.e., level I care), the CMMA provides real-time recording of multiple vital signs for each patient connected to his/her own Portable Transmit Receive Packs (PTRP), which is a portable, easily deployed rugged device. A “Quick Look” vital sign trend indicator feature enables first responders to check patient status at a glance, even in high ambient noise and low light conditions.

During continuing care from the first responder and transfer of care from the first responder to the emergency medical technician (EMT) care for transport to a medical facility (i.e., level II care), the PTRP records medication administration and the first responder/EMT added observation notes. The CMMA system is a spot check device that can notify the first responder/EMT of impending patient deterioration, especially from latent injuries such as tension pneumothorax, often encountered by first responders/EMTs in emergency situations. These conditions are difficult to detect in austere environments (high ambient noise and low light) using conventional means. The CMMA incorporates a data fusion algorithm that detects a deteriorating patient trend and notifies the first responder/EMT of this trend via front panel LED indicators. Also during the period of preparation for transport, the CMMA system facilitates patient triage and provides a quick and comprehensive medical records transfer between the first responder and the EMT.

At the medical facility (i.e., level III care), the CMMA system provides a 1-step “plug and play” review of the medication, first responder/EMT notes and vital sign history, when uploaded to a standard PC/lap-top with the analysis packaged in a format relevant to highly trained trauma surgeons without any special applications or viewers required to be pre-loaded on the standard PC/laptop. The battery operated PTRP unit records vital signs, observations, treatments and events for a minimum of 8 hours. Once the data is uploaded onto a host PC, one embodiment of PTRP 1 is cleaned, the nasal cannula and external water trap replaced, data cleared (by turning PTRP 1 off to data clear) and recharged for additional use. In another embodiment, the used PTRP 1 is discarded.

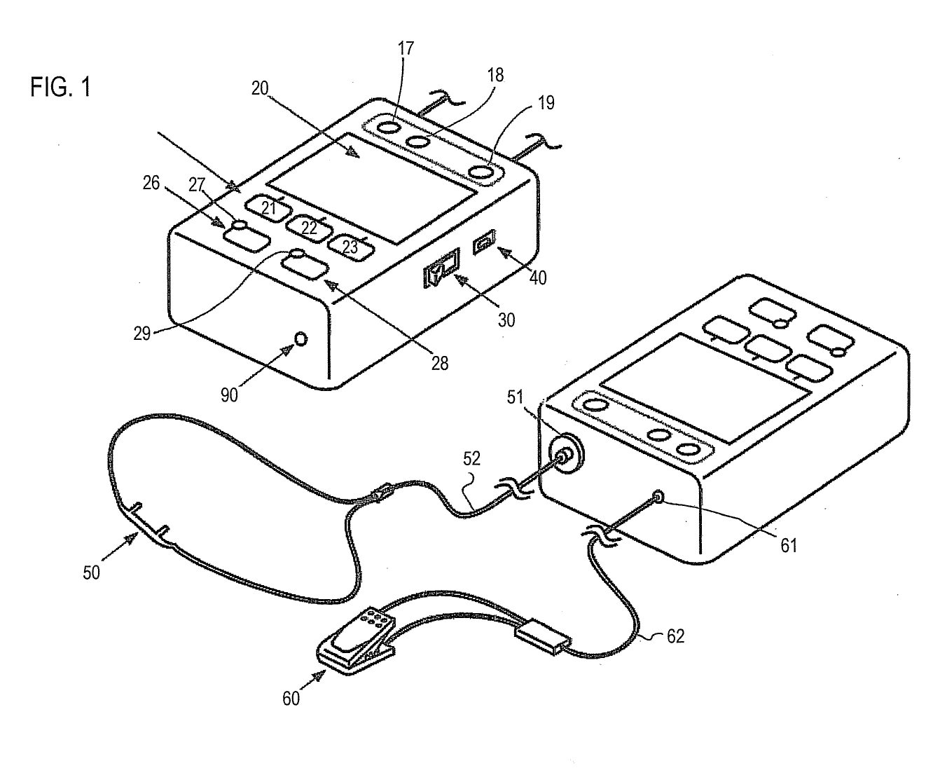

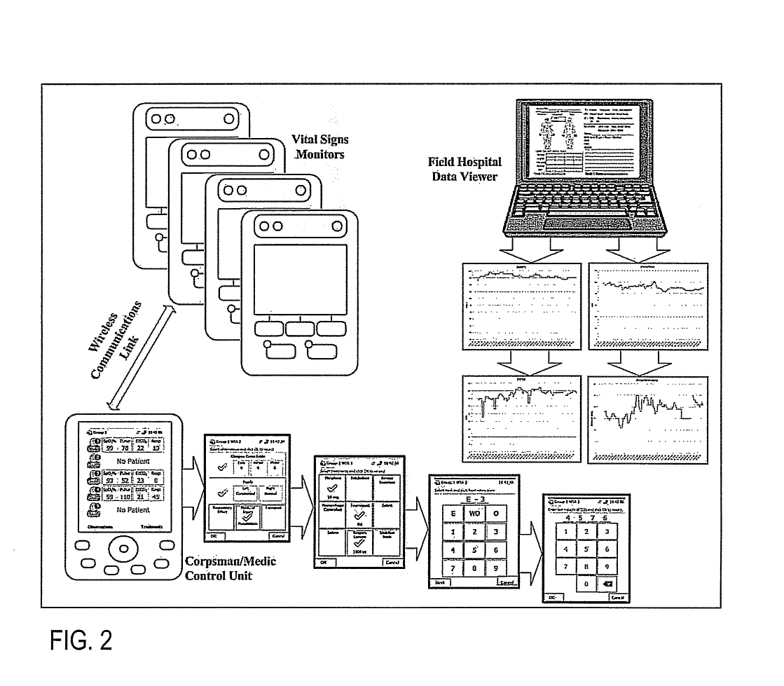

In the CMMA system, PTRPs can operate autonomously in a standalone mode or one or more PTRPs can be linked to one or more Corpsman/Medic Medical Assistant Control Units (CMMACUs) for situation involving multiple patients. The CMMA standalone system comprises one or more Portable Transmit Receive Packs (PTRPs), as shown in FIG. 1. The CMMA integrated system comprises one or more PTRPs and one or more CMMACUs, as shown in FIG. 2.

The CMMACUs are hand-held devices, such as PDAs, that wirelessly communicate with multiple PTRPs to provide a first responder the ability to monitor the vital signs and health trends of several patients while engaged in providing care to another patient. PTRPs also include at least one interface connector that is compatible with a standard personal computer/laptop computer interface for the transfer of data.

The system components of the CMMA are described in the following sections.

### Portable Transmit Receive Pack (PTRP)

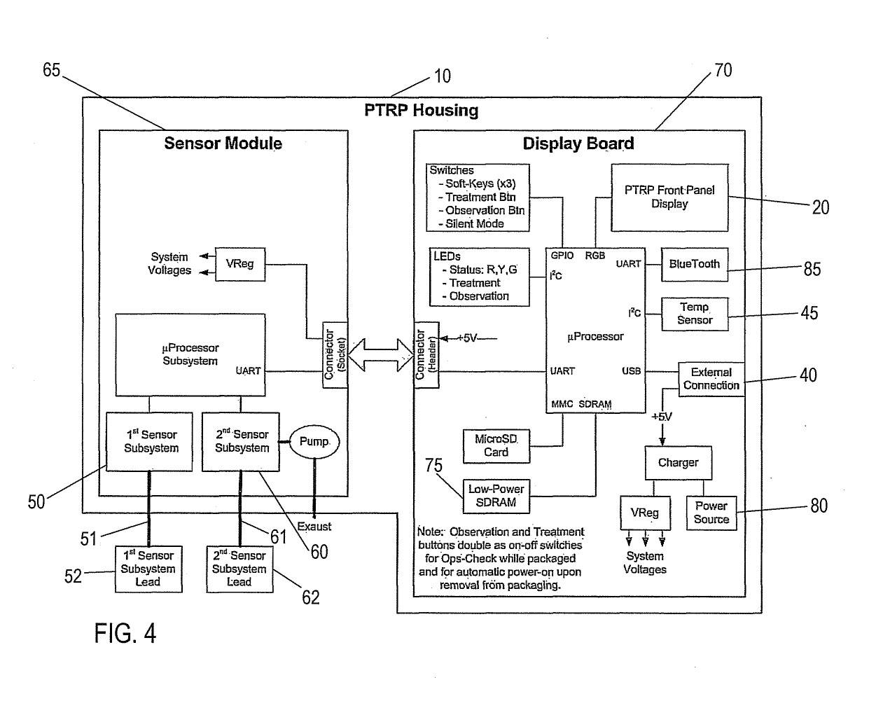

With reference to FIGS. 1-3, the PTRP 1 is a rugged, low cost, easy to use medical spot-check device for first responders to use in an emergency situation that automatically measures and records multiple physiological parameters which are relevant to first responders for the care and treatment of patients in an emergency situation. The PTRP 1 is a sealed unit that comprises PTRP housing 10, including PTRP front panel 15 containing a plurality of front panel indicators and a display 20, a PTRP operational mode switch 30, at least one connection port 40, such as a USB connector, and a first medical-grade (preferably FDA approved) physiological sensor subsystem 50 that connects to an external sensor via a first physiological sensor connector 51 on the PTRP, and a second medical-grade (preferably FDA approved) physiological sensor subsystem 60 that connects to an external sensor via a second physiological sensor connector 61 on the PTRP, as shown in FIG. 1. The two medical-grade physiological sensor subsystems 50 and 60 are capable of measuring and reporting the vital signs of the patient. With reference to FIG. 4, the PTRP 1 internally also includes at least one physiological sensor module 65, which can be a printed circuit board (PCB), a PTRP controller/display module 70, PTRP memory 75 for data storage, a power source 80, such as a battery, and an RF transceiver 85, such as a Bluetooth transmitter.

The PTRP housing 10 is fabricated from a lightweight and durable material, such as a hardened plastic or lightweight metal alloys that provide a sealable enclosure from environmental and liquid contaminants. The PTRP housing 10 can be rectangular in shape, as shown in FIG. 4, but is not constrained to be rectangular and its dimensions may be adjusted if the required packaged total volume decreases. The PTRP housing 10 is preferably EMI shielded, with the shielding acting as a continuous Faraday cage with the exception of the internal RF transceiver antenna (e.g., Bluetooth) which has an external RF access through the PTRP housing 10.

The PTRP housing 10 also includes an internal ambient temperature sensor 45, which is used for controlling the display drive characteristics and the ambient temperature waveform, which may optionally be logged in the data storage area of PTRP memory 75 for diagnostic purposes. The internal ambient temperature sensor 45 may be PCB mounted or PTRP housing mounted and is positioned as far away from internal thermal sources as possible, especially high-power devices such as the battery, the Capnography pump, if a Capnography sensor is installed in the PTRP, and the LCD backlight, if the display is an LCD. In one embodiment, PTRP 1 tracks ambient conditions that are pertinent to the patients condition, such as temperature, humidity, and atmospheric pressure.

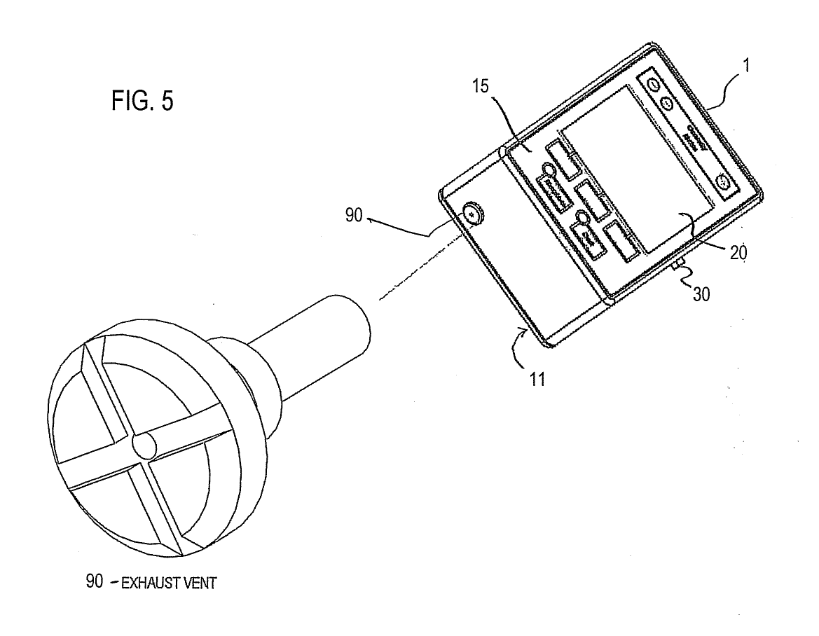

The PTRP housing has at least one connection port 40 that provides an external interface and power connector on one side surface of the PTRP housing 10. The connection port 51 for the first physiological sensor subsystem 50 and the connection port 61 for the second physiological sensor subsystem 60 are physically located on a different side surface of the PTRP housing 10 than connection port 40. In one embodiment, connection port 51 and connection port 61 are located on the top side of PTRP 1, when viewing PTRP front panel 15, to optimize the proximity of the sensor cables to the attachment points on the patient. The PTRP pneumatic exhaust vent 90 is physically located on another side surface of the PTRP housing 10, as shown in FIG. 5.

The pneumatic exhaust vent 90 attaches to the output of a pump of one of the physiological sensor subsystems 50 and 60 via a pneumatic tube. The exhaust vent 90 is permeable to gas, but provides a barrier to the ingress of liquids and solids into the PTRP 1. The pneumatic exhaust vent 90 includes vent fins to protect against clogging and provide positive or ambient pressure to the outside of the PTRP 1 (e.g. never negative pressure) block even with a finger pressed up against pneumatic exhaust vent 90, as shown in FIG. 5. Pneumatic exhaust vent 90 does not restrict PTRP orientation or grasping of PTRP 1 by the first responder/EMT or patient. No user interaction with PTRP 1 will block pneumatic exhaust vent 90 or impede operation of either first physiological sensor subsystem 50 or second physiological sensor subsystem 60.

The back surface 11 of the PTRP housing 10 is smooth to lay flat on the patient's chest with no interface connections or PTRP housing 10 features that would interfere with PTRP 1 lying on the patient's chest or being strapped to a gurney, for example|[drs1](e.g. no sharp points, posts, etc.). PTRP housing 10 does not restrict the positioning of PTRP 1 in relation to the patient.

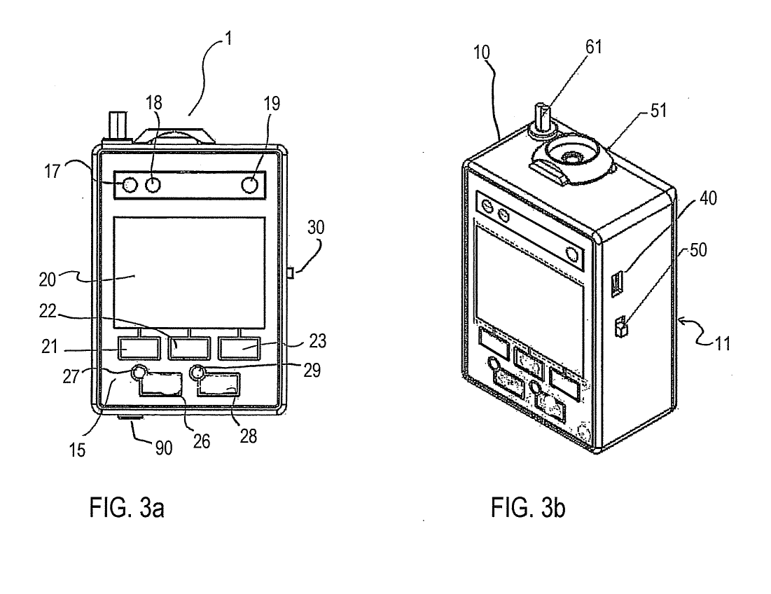

The PTRP front panel 15 is a sealed flat surface comprising three physiological trend indicators 17, 18 and 19, a PTRP display 20, such as an LCD display, at least one event key, such as 21, 22, and 23 located below the display 20, a treatment button 26, and an observation button 28, as shown in FIG. 3. The treatment button 26 and the observation button 28 are flush-mounted on PTRP front panel 15. The PTRP front panel 15 is completely covered with a continuous sheet of a printable material, such as Mylar, to seal the face and protect PTRP 1 against environmental ingress of liquids and solids. The PTRP front panel 15 also includes PTRP display 20, such as an LCD screen, which covers a significant portion of the front panel 15 of the PTRP 1.

The three physiological trend indicators 17, 18 and 19, are light emitting diodes (LEDs) that have three states: off, on-solid and on-blinking. When activated, each of the physiological trend indicators 17, 18 and 19 display a different colored light indicating the overall physiological trend of the patient. The three physiological trend indicators are arranged as a green physiological trend indicator 17, a yellow physiological trend indicator 18 and a red physiological trend indicator 19 in order from left to right across the upper portion of the PTRP front panel 15 above PTRP display 20. The red physiological trend indicator 19 is offset from the green physiological trend indicator 17 and the yellow physiological trend indicator 18 to emphasize to the first responder and/or EMT the importance of the red physiological trend indicator 19 and to facilitate red trend indicator receiving an appropriate level of attention/response from the first responder/EMT when lighted.

The treatment button 26 and an observation button 28 are used to receive and record inputs from the first responder and/or EMT transporting the patient to a medical facility. The treatment button 27 and the observation button 28 have at least the following capabilities:
Momentary (e.g. press to activate, release to de-activate);
Tactile feed-back to the user;
High probability of usage; and
Recording up to 10 presses maximum in typical usage for the treatment and observation buttons, in one embodiment of PTRP 1.

The treatment button 26 has a treatment light emitting diode (LED) indicator 27 that is located proximate to treatment button 26 and the observation button 28 has an observation LED indicator 29 that is located proximate to observation button 28 on PTRP front panel 15, as shown in FIG. 3. In one embodiment, the observation LED indicator 29 and the treatment LED indicator 27 are physically smaller than the physiological trend indicators 17-19 to emphasize the importance of the physiological trend indicators 17-19 and facilitate visual checking of the physiological trend indicators 17-19 at an emergency location (e.g., level 1 care). The treatment indicator 27 and the observation indicator 29 are in an “off” state upon completion of the PTRP system initialization. The treatment indicator 27 and the observation indicator 29 are activated by depressing the associated treatment button 26 or observation button 28, respectively. If the treatment indicator 27 and the observation indicator 29 are both activated, to minimize confusion to the first responder/EMT, treatment indicator 27 and observation indicator 29 blink synchronously such that the treatment and observation indicators turn on and turn off for the same time.

The PTRP operational mode switch 30 is physically located on the side of the PTRP housing and selects one of the at least two operational modes of the PTRP. For example, in one embodiment PTRP 1 has at least two operational modes: “Normal” and “Silent.” The default position when PTRP 1 is removed from its sealed packaging is “Normal” mode. When PTRP operational mode switch 30 is toggled to a second position, PTRP 1 changes to the “Silent” mode. The position of PTRP operational mode switch 30 determines the operational mode of PTRP 1 and is hard to change the position of PTRP operational mode switch 30 accidently, but the switch can be operated without use of any tools. If the PTRP operational mode switch 30 is inadvertently toggled to another position, PTRP operational mode switch 30 can be quickly and easily re-positioned to the desired position and the recording of patient data is not affected by the toggling of PTRP operational mode switch 30. The PTRP operational mode switch 30 may be mounted onto the PTRP housing or mounted directly on PTRP controller/display module 70.

The PTRP 1 includes at least one connection port 40, such as a standard mini-B style USB client port (mini USB), that is located on a side surface and exposed externally from PTRP housing 10. The connection port 40 is protected against environmental contaminants but is accessible for use without the use of tools (e.g. a removable plug when wrapped in the housing). The connection port 40 is covered with a removable protective plug and an icon label may be placed on the protective plug inserted into the connection port 40. The connection port 40 can be mounted to the PTRP controller/display module and if the connection port 40 is compromised (e.g. pins shorted together), PTRP 1 is capable of uninterrupted operation (excluding all connection port 40 functionality and charging). The PTRP displays the connection port status on PTRP display 20 as an icon.

For example, if the connection port 40 is a mini USB port, the mini USB port is labeled with a USB icon and the icon label may be on the protective plug. The mini USB connector can be mounted to the PTRP Controller/Display module 70 and if the USB connector is compromised (e.g. pins shorted together), PTRP 1 will provide uninterrupted operation (excluding all USB functionality and charging). The PTRP displays the USB connection status on PTRP display 20 as an icon.

The PTRP 1 includes an internal power source 80, such as a rechargeable battery, that provides power to operate PTRP 1 for at least 4 hours of independent, standalone operation. The internal power source 80 is completely contained within PTRP housing 10 and is loaded by only 20 uA while PTRP 1 is in the “packaged” state in sealed packaging 5.

The PTRP 1 includes PTRP memory 75 for storing a patient's vital sign data and medication and observation data from the initial connection of PTRP 1 to the patient. The PTRP's internal non-volatile memory stores vital sign data, waveforms, observation data and treatment data for a minimum of 8 hours of continuous data received at a 1 hertz update rate. The PTRP 1 can also receive and record data for one or more external physiological sensors that interface to PTRP 1 via connection port 40. PTRP 1 will display data from the one or more external physiological sensors and in some cases will use data from the one or more external physiological sensors in the PTRP physiological parameter analysis. Augmenting the physiological sensors of PTRP 1 with one or more external physiological sensors increases the accuracy of the PTRP reported trend status of the patient.

One example of an external device that can be attached to PTRP 1 is the ProPaq manufactured by Welch Allyn. The ProPaq is a popular monitor for medical transport that is flight approved in helicopters and supports a host interface that can be adapted to plug into PTRP connection port 40. ProPaq supports Non-Invasive Blood Pressure, ECG and temperature data collection from a patient. Once the ProPaq interfaces with PTRP 1, the ProPaq sends its captured physiological data to PTRP 1. The data from the ProPaq is stored and available with the data from the PTRP physiological sensors in PTRP memory 75 and all of the data stored in memory is available to the PC via the upload. This feature allows the medical personnel to view more data relevant to the patient's condition without having to change the PTRP hardware configuration.

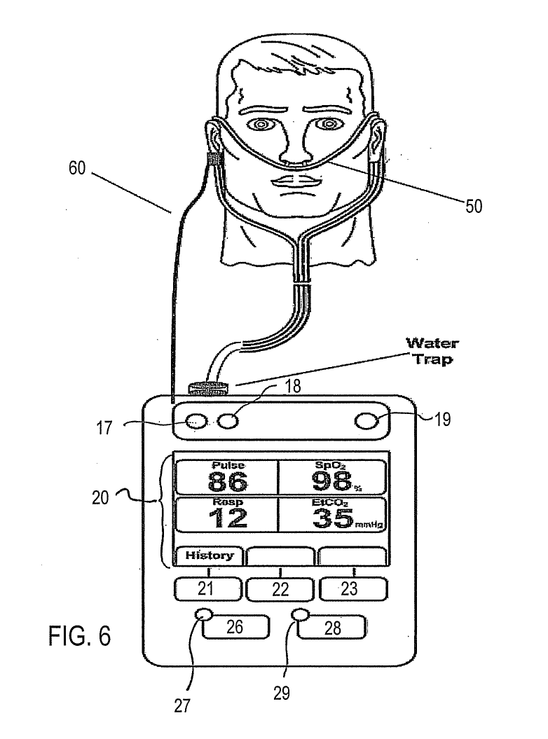

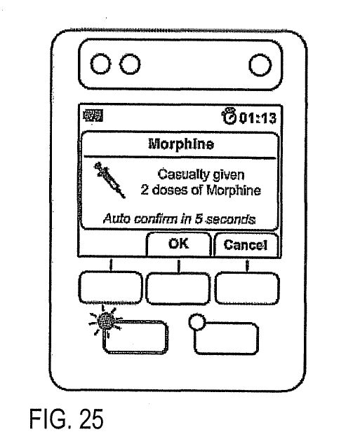

In the event the data log in PTRP memory 75 is completely full with data (e.g. a recording longer than the maximum storage space), PTRP 1 will continue to operate the physiological trend indicators but will stop recording vital sign data because medical history near the time of an incident is more valuable than medical history subsequent to the incident. The PTRP 1 will also alert the first responder/EMT that PTRP memory 75 is full on PTRP display 20 and via CMMACU 100 (shown in FIG. 25). FIG. 6 shows the connection of one embodiment of PTRP 1 to the patient.

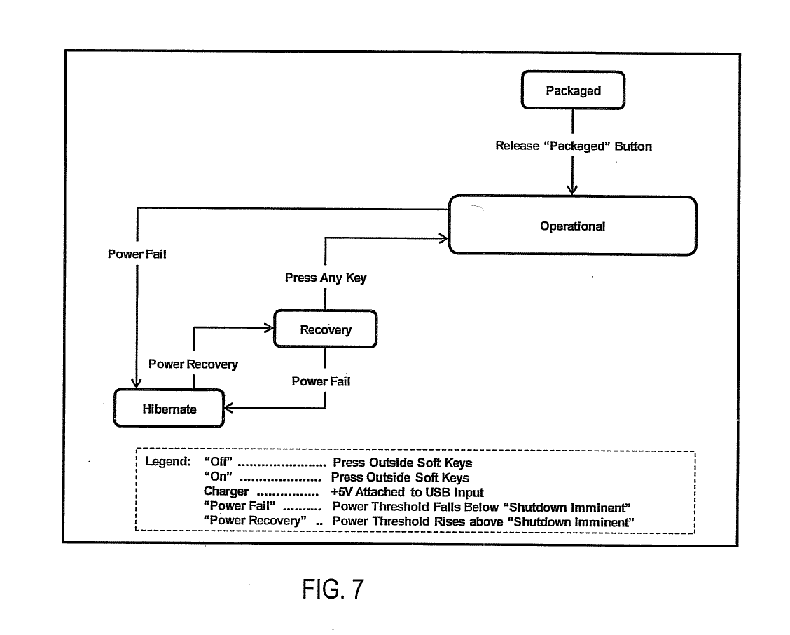

The PTRP 1 has at least four (4) system states; Packaged, Operational, Hibernate, and Recovery, as shown in FIG. 7. The PTRP 1 will be deployed in the “Packaged” state and very likely remain in that state for an extended period of time. PTRP 1 is in a zero power state when “packaged”. When PTRP 1 is unpackaged and automatically initializes, PTRP 1 transitions to the “Operational” state and remains in that state until the internal power source 80 is no longer capable of providing power for the PTRP operational state (e.g., low battery power, shutdown imminent). The “Hibernate” state is a very low power state that PTRP 1 enters automatically in order to preserve the data recorded in PTRP memory 75. The recovery state is also a low-power state but supports the download of data from PTRP memory 75 to a PC. If the internal power source is completely drained, PTRP 1 retains the data in memory and will recover when power is applied from an external source.

The PTRP 1 and its associated physiological sensor cables, 51 and 61, are initially enclosed in a heat shrunk plastic sealed packaging. The heat shrunk plastic sealed packaging opens in a single step, hand tear operation. The sealed packaging protects PTRP 1 from the stresses of being stored and carried by first responders for an extended period of time (e.g., 18 months). The sealed packaging ensures that the physiological sensor cables, 51 and 61, are consistently dressed with no stress points and when unpackaged, physiological sensor cables 51 and 61, are not prone to tangle, knotting or detachment from PTRP 1. Permanently attached and integral to sealed packaging is an “OFF” switch or clip that keeps PTRP 1 in an “OFF” state during shipping and storage in the sealed packaging. The “OFF” switch or clip is automatically removed from PTRP 1 when a user opens the sealed packaging and removes PTRP 1 from the opened packaging. When the sealed packaging is opened, PTRP 1 automatically powers up without any user input.

In the sealed packaging, PTRP 1 has two distinct operational states: (i) an “OFF” state in which PTRP 1 remains in the lowest possible power consumption state, and (ii) running an operational check (Ops Check) of the internal systems in response to user inputs while within sealed packaging.

While PTRP 1 is sealed within the sealed packaging in an “OFF” state, PTRP 1 conserves power by not powering the PTRP controller/display module 70, including the microprocessor, as well as the RF transceiver 85, the physiological sensor subsystems, 50 and 60, the physiological trend indicators, 17-19, and PTRP display 20 on PTRP front panel 15. The only components in PTRP 1 that are powered in the “OFF” state are the circuits that detect the “OFF” switch or clip and the treatment and observation buttons that a user depresses to initiate the PTRP “Ops Check.”

While in the storage and shipping configuration in the sealed packaging, PTRP 1 supports a PTRP “Ops Check” capability. Running an Ops Check does not compromise the packaging. The Ops Check is initiated by pressing and holding the two front panel treatment and observation buttons simultaneously. Upon initiation of the Ops Check, PTRP 1 automatically performs a check of PTRP display 20, physiological trend indicators, 17-19, and physiological sensor subsystems 50 and 60. The PTRP display 20 initially displays an “in progress” screen and upon completion of the Ops Check, the PTRP display 20 displays an Ops Check Pass or Fail to the user. The Ops Check “Pass/Fail” status on PTRP display 20 and all indicators on PTRP front panel 15 are visible to the user when packaged.

In the process of removing the PTRP from the packaging, the “OFF” switch holding the PTRP 1 in the “Packaged” state is released and the power source provides power to the PTRP controller/display module 70, which includes a microprocessor. Upon receiving power, PTRP controller/display module 70 initiates the boot process to initialize PTRP 1, which includes a power check and verification that the release of the “Packaged” button was not a false input due to a transient input (e.g. a momentary release due to mechanical stress). During PTRP initialization (e.g. the “Boot” process) each of physiological trend indicators 17-19, treatment button 26 and observation button 27 synchronously blink twice (¼ second on, ¼ second off ×2) and an initialization screen is displayed on the display.

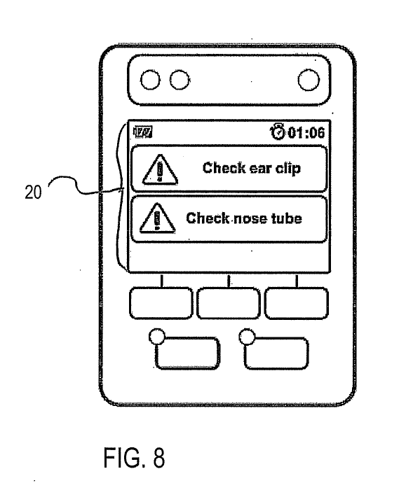

During initialization, the initialization screen shows the elapsed time as “00:00” in the upper right portion of PTRP display 20 and PTRP power source 80 is displayed as full (i.e., a full battery icon) in the upper left portion of PTRP display 20, as shown in FIG. 8. As the PTRP 1 initializes, various regions of the initialization screen of PTRP display 20 becomes active (e.g. the “full” battery icon is replaced with actual battery status, timer starts counting up, etc.). The entire boot process for PTRP 1 is completed and physiological sensor sub-systems 50 and 60 begin generating valid sensor status in less than 10 seconds. It is important to note that 10 seconds after unpackaging PTRP 1, a valid physiological sensor status may be a sensor fault because physiological sensor sub-system leads 52 and 62 have not been attached to a patient, but this sensor fault is still considered a valid status. In the event that PTRP 1 fails the initialization self-check, PTRP 1 will enter the “Hibernate” state.

After removing PTRP 1 from its packaging, the first responder/EMT connects one end of each of the physiological sensor sub-system leads 52 and 62 to PTRP connection ports 51 and 61 and the other end to the patient. PTRP 1 can be connected to the patient while the PTRP is initializing. After connecting the PTRP physiological sensor sub-system leads 52 and 62 to PTRP 1 and the patient, the PTRP is placed on the patient's chest, placed on the ground beside the patient, or attached to the gurney or stretcher on which the patient is lying. The PTRP 1 can be connected to the patient, gurney or stretcher using double sided adhesive tape attached to the back surface 11 of the PTRP housing 10 or using a clip that attaches PTRP 1 to the gurney or stretcher. The PTRP 1 can also be worn by the patient by wearing PTRP 1 on a lanyard attached around the patient's neck or in a backpack on the patient's back, for example. The PTRP 1 will collect, record and display patient vital sign data and physiological trend status. If PTRP 1 is operating in the integrated CMMA system, PTRP 1 will also be transmitting patient vital sign data and physiological trend status to a CMMACU 100 and receiving and storing data from CMMACU 100.

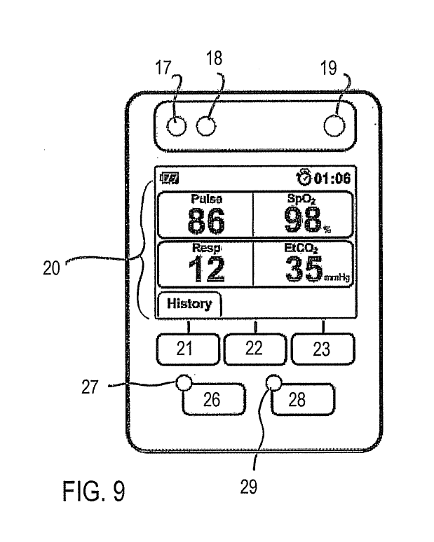

The PTRP 1 includes a PTRP display 20, such as an LCD screen, that enables direct numerical reading of multiple vital signs being measured by PTRP 1 on the main display screen, as shown in FIG. 9. Additionally, the main display screen on PTRP display 20 includes at least the following indicators:
An elapsed-time counter 32 that counts time in hours and minutes from 00:00 at PTRP initialization. In one embodiment, PTRP 1 is capable of displaying time up to 999:59 hours. The elapsed time counter 32 can provide real-time date and time information when synchronized with a PC. The elapsed time counter 32 is continuously displayed whenever PTRP display 20 is active. The elapsed time count is accurate to within 1 second per hour (0.02% accuracy). This timer is necessary to record times that treatments were administered. In another embodiment, PTRP 1 displays real-time after initialization. In this embodiment, PTRP 1 contains means for determining real-time, such as an integrated GPS chip.
PTRP power level indicator 81 that uses a battery icon to graphically depict the status of the PTRP power source 80.
RF link status indicator 86 which depicts whether the RF transmitter 85 is connected to an CMMACU 100 or is operating standalone and the RF link is not connected;
Three event keys 21-23 are provided on the lower portion of PTRP display 20. In one embodiment, the three event key buttons 21-23 are, from left to right, History event key 21, OK event key 22 and CANCEL event key 23, as shown in FIG. 9.

The PTRP 1 supports at least the eleven (11) capabilities shown in Table 1. Some of the capabilities are supported in multiple PTRP states (e.g. Power Scavenging is supported in Operational, Recovery and Hibernate states), while other features are only supported in one PTRP state (e.g. “Quick Look” patient trend indicator status is only supported in the “Operational” state while in the “Normal” mode), as shown in Table 1.

TABLE 1
PTRP Capabilities in Each PTRP State

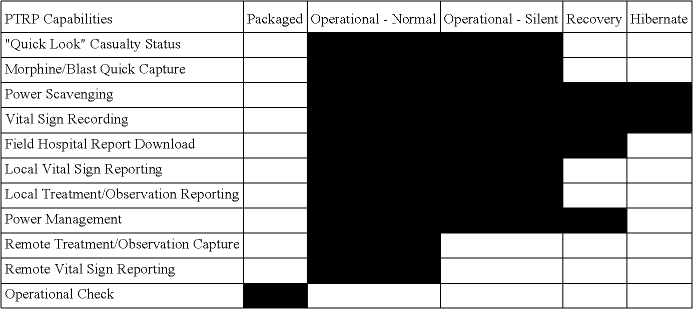

The PTRP 1 displays first responder/EMT observations and first responder/EMT treatments entered into PTRP 1 on PTRP Display 20 when the first responder/EMT depresses the History event key 21. Upon depressing the “History” event key 21, the history display screen is displayed on PTRP display 20. The history display screen will timeout in 5 seconds and revert to the main screen on PTRP display 20.

In the operational mode, PTRP 1 has at least two modes of operation: “Normal” mode or “Silent” mode. After PTRP initialization, the default mode of PTRP operation is “Normal” mode. In “Normal” mode, PTRP 1 displays the three physiological trend indicators 17-19 on PTRP display 20, Treatment indicator 27 and Observation indicator 29 are available for information display and user feed-back on the front panel, as shown in FIG. 9. Additionally, in some embodiments, RF transmitter 85 is fully powered and capable of communicating with one or more CMMACUs 100, as indicated by RF link status indicator 86 on PTRP Display 20.

When PTRP 1 is operating in “Normal” mode after PTRP physiological sensor sub-system leads 52 and 62 are connected to PTRP 1 and a patient, The PTRP 1 uses a least lower bound thresholding algorithm to determine the patient's physiological trend status and illuminates the appropriate physiological trend indicators 17-19 to indicate the algorithmic result of the physiological parameter analysis performed by PTRP 1. The three physiological trend indicators 17-19 provide a “quick look” physiological trend status of the patient to the first responder/EMT.

In this embodiment, the current condition of the patient is indicated by one of the physiological trend indicators being illuminated as a solid light (continuous “On” position). A change in the current condition of the patient (i.e., trend) is indicated by a second physiological trend indicator being illuminated as a flashing light (flashing “On” position) for a period of time while the current physiological trend indicator remains lit as a solid light. The second physiological trend indicator then transitions to a solid light and the first physiological trend indicator is turned off. This actuation scheme permits the physiological trend indicators to exhibit a controlled manner of behavior rather than blink erratically in response to sensor “glitches”, such as a loose connection.

For example, a continuous green physiological trend indicator 17 and a flashing yellow physiological trend indicator 18 indicates the patient's condition is deteriorating, whereas a continuous yellow physiological trend indicator 18 and a flashing green physiological trend indicator 17 indicates patient's condition is improving. The transitions from one state (e.g., green trend indicator) to another (e.g., yellow trend indicator) are more important to first responder/EMTs and clinicians than steady state conditions.

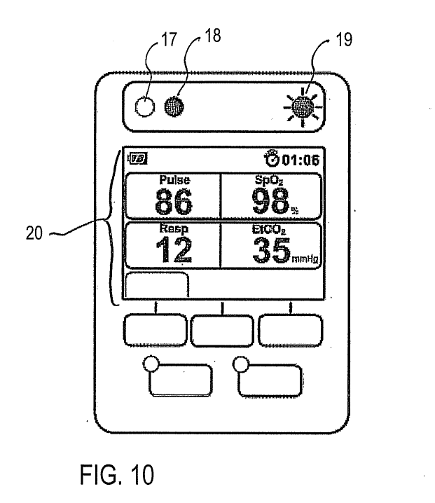

For example, if three of the displayed numeric sensor values on PTRP display 20 are in the yellow range while the other displayed sensor value is trending into the red range, the trending sensor value will eventually “tip” the PTRP 1 to transition the displayed trend indicator from yellow physiological trend indicator to red physiological trend indicator 19, as shown in FIG. 10. The red physiological trend indicator 19 will remain lit until the “red” trending sensor value trends toward the “yellow” range and the other sensor values remain in the “yellow” range, which will result in PTRP display 20 displaying yellow physiological trend indicator 18. The trending algorithm weights the trending physiological sensor, whether trending lower or higher, and transitions to the appropriate trend status indicator over a period of time. The PTRP 1 does not skip over yellow physiological trend indicator 18, even if there is a sudden change in the patient's health sufficient to transition from green physiological trend indicator 17 directly to red physiological trend indicator 19. The PTRP 1 will display yellow physiological trend indicator 18 for the minimum time and then transition to red physiological trend indicator 19. Red physiological trend indicator 19 is physically offset from yellow physiological trend indicator 18 and green physiological trend indicator 17 to visibly indicate a dangerous patient condition regardless of orientation of the first responder/EMT to PTRP 1, as shown in FIG. 10. Additionally, the offset position of the red physiological trend indicator 19 allows observation by the first responder/EMT under low light or under conditions where the first responder/EMT is wearing night vision goggles.

In the operational state, if PTRP display 20 is an LCD, PTRP 1 will constantly power the LCD backlight so that the first responder/EMT does not need to physically interact with PTRP 1 just to bring a dimmed backlight back up to full visibility to “glance” at the displayed vital sign data of the patient.

If the first responder/EMT toggles the operational mode switch 30 on the side face of PTRP 1, PTRP 1 is switched to a “Silent” mode of operation. The “Silent” mode minimizes the visible light, RF energy and audible noise generated by PTRP 1 for situations where PTRP 1 emissions interfere with a patient resting or where first providers and patients need to hide from a threat. When the operational mode switch 30 is toggled to the “Silent” mode, PTRP 1 turns off the three physiological trend indicators 17-19, Treatment indicator 27 and Observations indicator 29 on PTRP front panel 15, turns off RF Transmitter 85, switches PTRP display 20 to display colors to red on black, and dims the backlight such that PTRP display 20 is readable in a dark room. The RF link status indicator 86 changes to link disabled when in “Silent” mode. In the “Silent” mode, physiological sensor subsystems 50 and 60 are fully operational, retaining full vital sign data collection at the default rate of 1 Hz. and retaining full Treatment and Observation buttons 26 and 28 data collection and recording. The first responder/EMT can toggle PTRP operational mode switch 30 to normal to check the displayed physiological sensor data and then toggle back to “silent” mode. PTRP 1 operations affected by switching to the “Silent” mode, including turn off of PRTP display 20, physiological trend indicators 17-19, Treatment and Observations indicators 27 and 29 and RF transmitter 85, are executed in less than 1 second. PTRP operations not affected by “Silent” mode continue uninterrupted.

The PTRP 1 will perform at full functionality, collecting and displaying patient vital sign data in the operational state until PTRP power source 80 (e.g., battery) reaches the “Shutdown Imminent” power condition. The PTRP 1 monitors the level of PTRP power source 80, displays PTRP power level indicator 86 on PTRP display 20 and measures power usage to predict the onset of the “Shutdown Imminent” power condition and ensure the successful transition of PTRP 1 to the “Hibernate” state with no loss of data and no loss of elapsed time. The PTRP 1 power level warnings and power recharging options are explained later in this document.

The PTRP 1 also supports a “Hibernate” state which is only entered from the “Operational” state when a power failure is imminent (e.g., the battery drains to the “Shutdown Imminent” level). The threshold for “Shutdown Imminent” will provide enough power for the microprocessor in PTRP Controller/Display module 70 to perform an orderly shut-down of PTRP 1. If PTRP power source 80 is below the “Shutdown Imminent” power level, PTRP 1 will remain in the “Hibernate” state until PTRP 1 has sufficient power to enter the “Operational” state. The primary function of the “Hibernate” state is to preserve the integrity of the data log when power is dangerously low. The PTRP 1 can remain in “Hibernate” state for a minimum of 3 days (72 hours) without losing any data stored in PTRP memory 75.

In the “Hibernate” state, the microprocessor in PTRP Controller/Display module 70 is fully powered and in control of power distribution throughout PTRP 1. The PTRP 1 sheds the power loads of physiological sensor sub-systems 50 and 60, RF transmitter 85, PTRP front panel display 20. In the case of the power level dropping below the “Shutdown Imminent” threshold, PTRP 1 will continue to track elapsed time as long as there is sufficient power to keep the microprocessor in PTRP Controller/Display module 70 running in “sleep” mode in the “Hibernate” state. The microprocessor may wake as needed in the “Hibernate” state, but will not enable power to physiological sensor sub-systems 50 and 60, RF transmitter 85, PTRP display 20 and the integrity of the data stored in memory is preserved.

If the first responder/EMT or hospital personnel plug PTRP 1 into an external power source, PTRP 1 will monitor the power source charge state and when sufficient power is available, PTRP 1 will transition to the “Recovery” state. When PTRP 1 enters the Recovery” state, PTRP 1 will continue to charge power source 80. When PTRP 1 transitions from “hibernate” state to either the “Recovery” state or the “Operational” state, PTRP 1 will resume logging data into memory 75 and support uploading data currently in memory 75. The data in memory 75 will show a gap in the data, but there will be no loss of data continuity.

The PTRP 1 also includes a “Recovery” state, which provides an efficient recharge cycle for PTRP 1. The PTRP 1 will enter the “Recovery” state from the “Operational” state if PTRP 1 detects inactivity for 3 minutes with the charger attached (e.g. the first responder/EMT or hospital personnel plugged the PTRP into a power source and walked away with the charger attached). “Inactivity” is defined as:

Radio disconnected;

Physiological sensors disconnected (e.g. not attached to a patient); and

No user interaction (e.g. no button presses).

The PTRP 1 exits the “Recovery” state and enters the “Operational” state upon any first responder/EMT or hospital personnel input including:

Any button press;

Any Toggle of PTRP operational Mode switch 30; or

The removal of the external power source from connection port 40.

The PTRP 1 will perform an “Ops Check” whenever transitioning from the “Recovery” state to the “Operational” state. If there is insufficient power for PTRP 1 to advance to the “Operational” state, PTRP 1 will remain in the “Recovery” state. If there is insufficient power to remain in the “Recovery” state (e.g. the first responder/EMT or hospital personnel removed the USB charger, the lap-top went to sleep, etc.) PTRP 1 will transition to the “Hibernate” state.

In the Recovery state, red physiological trend indicator 19 will blink for ¼ second every 20 seconds, indicating that PTRP 1 is active. If the power source 80 becomes fully charged, red physiological trend indicator 19 will increase the blink rate to 4 times the above rate (e.g. off for 5 seconds rather than 20). All connection port 40 functionality is supported while in the “Recovery” state. If the PC interfacing with PTRP 1 via connection port 40 generates a “Power Down” command (e.g. a lap-top going to sleep), PTRP Twill remain in the “Recovery” state, but if power source 80 drops below the “Full” level, red physiological trend indicator 19 will return to blinking at the slower rate.

The PTRP 1 uses a combination of PTRP electronics and PTRP software to manage all power sources, power loads and state transitions in PTRP 1, including battery draining, battery charging and transition between states based on power levels. The PTRP 1 continuously displays a power level indicator 86 on PTRP display 20 whenever PTRP display 20 is powered. The PTRP 1 can unambiguously distinguish between at least the following levels of power status:
Full
¾ Full
½ Full (aka. ½ Empty)
¼ Full
Shutdown Imminent (sufficient power to successfully enter/exit “Hibernate” state)
External Power, Charging
External Power, not Charging

The power level indicator 85 on PTRP display 20 indicates the amount of charge in power source 80, even while charging. If the external power source is insufficient to supply enough power for PTRP 1 to fully operate in the “operational” mode and charge power source 80 simultaneously, power level indicator 86 will display the presence of the external power source, but continue to show that the power level of power source 80 is decreasing.

Based upon a series of internal self checks that are transparent to the first responder/EMT, PTRP 1 detects an upcoming power failure and provides a pop-up warning/alert when the fuel level drops from ½ Full to ¼ Empty to the first responder/EMT to recharge PTRP 1 if possible. The PTRP display 20 shows the battery low pop-up warning/alert message at predetermined time intervals, which are associated with the rate of battery depletion) and PTRP 1 requires either the first responder/EMT to depress OK event key 22 to clear the pop-up warning/alert message from PTRP display 20 or to plug in a power charger. If the first responder/EMT does not dismiss the pop-up warning/alert message, PTRP 1 will continue to display the pop-up warning/alert message. If the pop-up warning/alert message has been dismissed, a new pop-up warning/alert message will appear every 15 minutes until PTRP 1 reaches the “Shutdown Imminent” power level. If the first responder/EMT plugs a power charger into PTRP 1 while the power level of PTRP 1 is in the “¼ full warning region” of power source 80 and the charger is sufficient to sustain the current “state” of PTRP 1 and charge power source 80, further low power pop-up warning/alert messages will be suppressed by the PTRP. The pop-up warning/alert messages will be automatically dismissed when the first responder/EMT plugs PTRP 1 into a power supply capable of sustaining the “Operational” state while the pop-up warning/alert message is displayed on PTRP 1. The automatic detection of an external power supply and automatic dismissal of the pop-up warnings enables PTRP 1 to begin collecting and displaying patient vital sign data with fewer user required operational steps.

If the first responder/EMT unplugs/removes the power charger from PTRP 1 while the power level is in the ¼ full region, PTRP 1 immediately displays the low-power pop-up warning/alert message. Once power is insufficient to remain in the “Operational” state (e.g. reached the “Shutdown Imminent” power level), PTRP 1 will enter the “Hibernate” state.

External power input to PTRP 1 is provided via connection port 40, such as a mini USB connector, on the side surface of PTRP housing 10. If connection port 40 is a mini USB connector, the external power input must be within the +5V specification of a standard USB port. The PTRP 1 is tolerant of excessive current on connection port 40 to support charging from a power source that is not a PC.

The PTRP 1 is also capable of receiving power from a “power only” connection that is not sourced by an active PC host. The PTRP 1 will detect the presence of a “power only” connection at connection port 40 and fully support all modes of operation when the “power only” cable is inserted, removed and especially if inconsistent power is available on the cable (e.g. cable sourced by a solar panel that fades whenever the light source fails).

Additionally, each PTRP 1 includes a “Hand Off” data capability in which a PTRP 1 with a critically low power source will transfer the data stored in memory 75 to another PTRP 1 having a greater level of power in its power source 80 (e.g., an unused “fresh” PTRP). In this mode, the low power PTRP 1 is connected, via connection port 40 to a “fresh” PTRP 1 and the “fresh” PTRP 1 provides a momentary level of charge, sufficient to automatically transfer data from memory 75 in the low power charge PTRP 1 and concatenate data from low power charge PTRP 1 and “fresh” PTRP 1 in memory 75 of “fresh” PTRP 1.

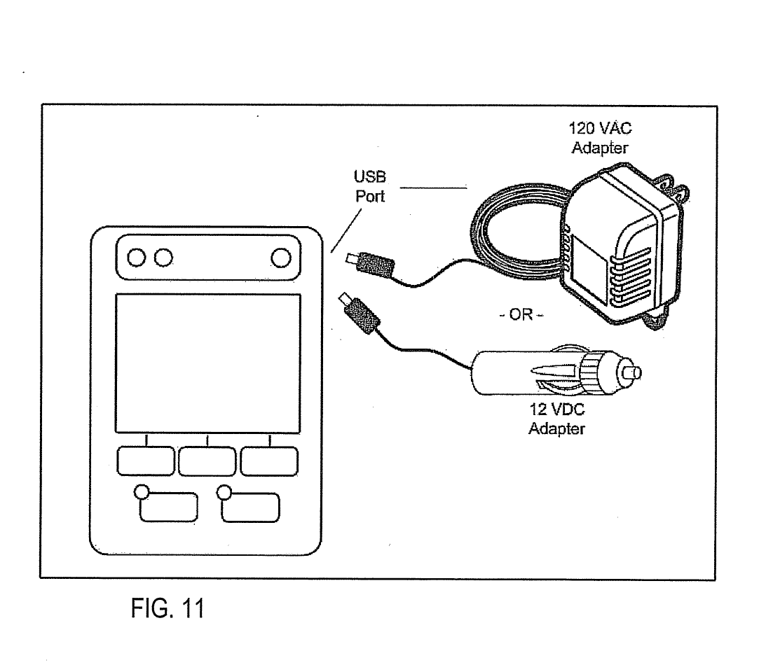

The PTRP 1 also can charge directly from an external regulated power source to 5 VDC. To charge from an external regulated power source, PTRP 1 is connected to the external regulated power source via the connection port 40. In some embodiments, PTRP 1 is equipped with a 12 VDC to 5 VDC power regulator that enables PTRP 1 to charge from a standard automobile/utility 12 VD receptacle, as shown in FIG. 11.

Additionally PTRP 1 has the capability to “Scavenge” power from potential sources other than a conventional 12 VDC utility receptacle these include, direct connection, using field configured adapters, to a 12 VDC battery, an 18′VDC solar panels, or other existing battery power sources, such as standard field radio communications batteries and aircraft 24 VDC sources. The use of these other sources is defined as the ability to “Scavenge” power for extended operations beyond the typical time provided with the PTRP standard power source.

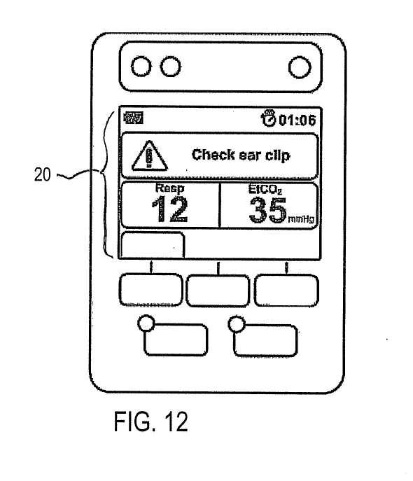

If one of the physiological sensor subsystems, 50 or 60, generates an error rather than valid vital sign data, PTRP 1 will immediately transition to the related fault display in lieu of displaying “stale” vital sign data. When one of the physiological sensor subsystems, 50 or 60, of PTRP 1 is faulted, the PTRP physiological trend algorithm gracefully degrades and physiological trend indicators 17-19 will continue to show the patient's status based on the physiological sensor data from the remaining sensor subsystem. In the case of single sensor fault, existing vital sign information will be used to influence physiological trend indicators 17-19 and an information/attention icon will be displayed over the numeric values area of PTRP display 20 that are associated with the faulted physiological sensor subsystem. PTRP 1 ceases displaying numeric vital sign data affected by the faulted physiological sensor subsystem but continues to display numeric vital sign data that is not affected by the faulted physiological sensor subsystem. For example, a single fault case of a nasal cannula fault (clog, detachment, etc.) is shown in FIG. 12. In the example shown, the numeric values for respiratory rate and EtCO2 are not displayed and the information/attention icon “check nose tube” is displayed over the display area the numeric values for respiratory rate and EtCO2 are normally displayed, the displayed pulse rate has crossed the “red” threshold to 110 beats per minute (BPM) and the patient physiological trend indicator is transitioning from “yellow” to “red”.

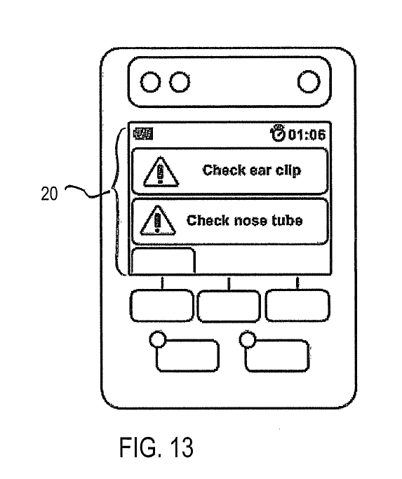

If both of the physiological sensor subsystems, 50 or 60, are faulted and there is no physiological data available to the algorithm, an information/attention icon will be displayed over the numeric values area of PTRP display 20 and all three physiological trend indicators 17-19 are turned off, as shown in FIG. 13.

PTRP 1 reports faults by displaying only immediately useful information to the first responder/EMT so that the first responder/EMT can take appropriate action. In the above embodiment, actions that can be taken at the site of the emergency to correct sensor faults are limited to:
Replace PTRP 1;
Reattach physiological sensor leads 52 and 62 (for example, nasal cannula and/or SpO2 clip); or
Unclog PTRP connection port 51 or 61 (for example, CO2 line, nasal cannula, water trap and/or exhaust vent).
   PTRP 1 does not display errors using error codes or cryptic error source references that would only be a distraction to first responders/EMTs at the site of the emergency.

If the patient is dead prior to PTRP 1 attachment or if the patient dies while attached to PTRP 1, PTRP 1 will either show “Fault” on the sensors or a notification that the sensors need to be checked (e.g. the sensors are no longer attached to a viable patient). In one embodiment, PTRP 1 records medication provided during CPR and resuscitation efforts.

The PTPR 1 supports the download of the recorded vital signs and observations captured in the PTRP data log in memory 75 to a PC over connection port 40, such as a standard USB cable. The PC does not need to be connected to the Internet, nor have any PTRP 1 specific software pre-loaded onto it. Within PTRP 1 resides a comprehensive set of software macros (e.g., Microsoft Excel and/or Java) required to download the information to a PC; no pre-loading of software is required for the interfacing hospital PC.

For example, if the connection port 40 is a mini USB connector, the PC that interacts with PTRP 1 must be fully USB compliant, at a minimum certification level of USB 1.1 (preferable USB 2.0 or greater), be capable of recognizing the attachment of a mass storage device over USB and will open and execute an Excel 97 formatted template. The PC is only requires the USB drivers pre-packaged in the PC. The PTRP 1 interfaces directly to a standard USB port on the PC and the information is automatically downloaded into a spreadsheet. The PTRP-PC data download operation for the example above has 3 basic steps;
1) Connect PTRP 1 to the PC via a standard USB cable (mini-B at the PTRP end). The PTRP 1 connects to the PC via USB such that it can automatically pop up a browser with a single file in it if the PC has not disabled the automatic operation. The single file is a template file for a standard data format such as Microsoft Excel 97 template file. Java, html and other standard data formats can be used. The PTRP 1 will display no other files and the drive is read-only.
2) Double-Click on the standard data file, such as an .xlt file. The only action required to view the data stored in PTRP memory 75 is to double-click on the file. Once this occurs, the PC will auto-launch the appropriate program, such as Excel and PTRP 1 will show the entire data set to the application. The file will open in less than 10 seconds on a conventional lap-top or desktop PC.
3) Save the data to the local PC. The user should save the data file to the PC's local drive. The user may either choose “Save As” or simply exit the program. Either action will bring up the usual browser, which is pre-populated with the default file name if no patient identification data was entered, or with the patient's ID. The path name defaults to either the “my data” folder or the folder which was last used to store a PTRP-generated patient record. If there is no history of saves available, the path name defaults to “My Data.”

The excel file itself is unlocked and may be edited by the user. However, no edits made by the user are “pushed” down and stored by PTRP 1. The PTRP 1 preserves the data throughout the transfer process described above, and continues to track elapsed time and/or log data into the data log in PTRP memory 75 throughout this process. If at some future point a USB device requests the data from PTRP 1, the entire data log in PTRP memory 75, including all data captured since the last transfer is transferred to the PC and sensor fault information in PTRP memory 75, as described above.

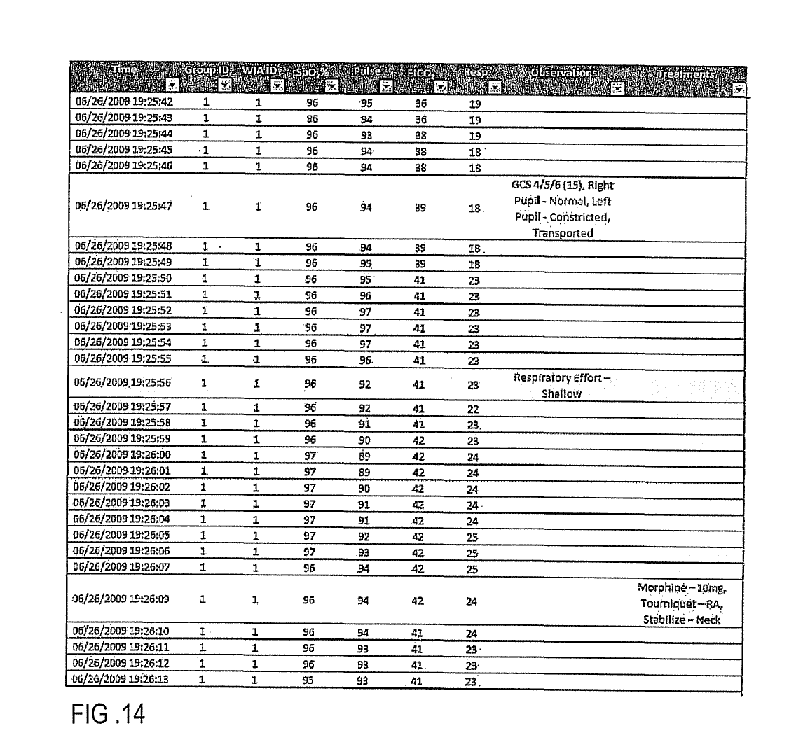

Upon connection to the hospital PC, the PTRP synchronizes its on-board elapsed time to the PC's real-time clock to calculate the overall real-time of the PTRP's data since it was turned on and connected to the patient. The PTRP also automatically formats the raw patient vital sign, observation and medication information into an Excel spreadsheet format for printing, as shown in FIG. 14.

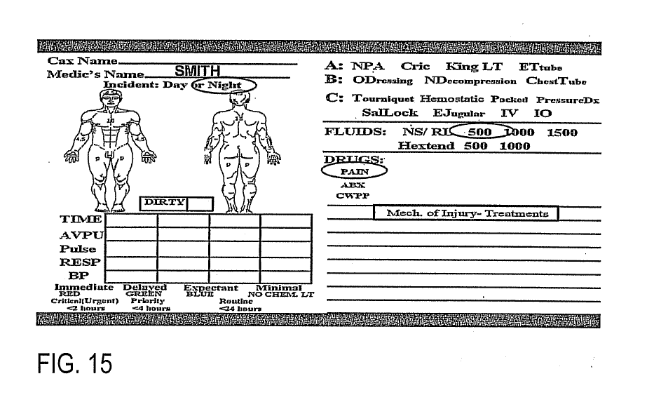

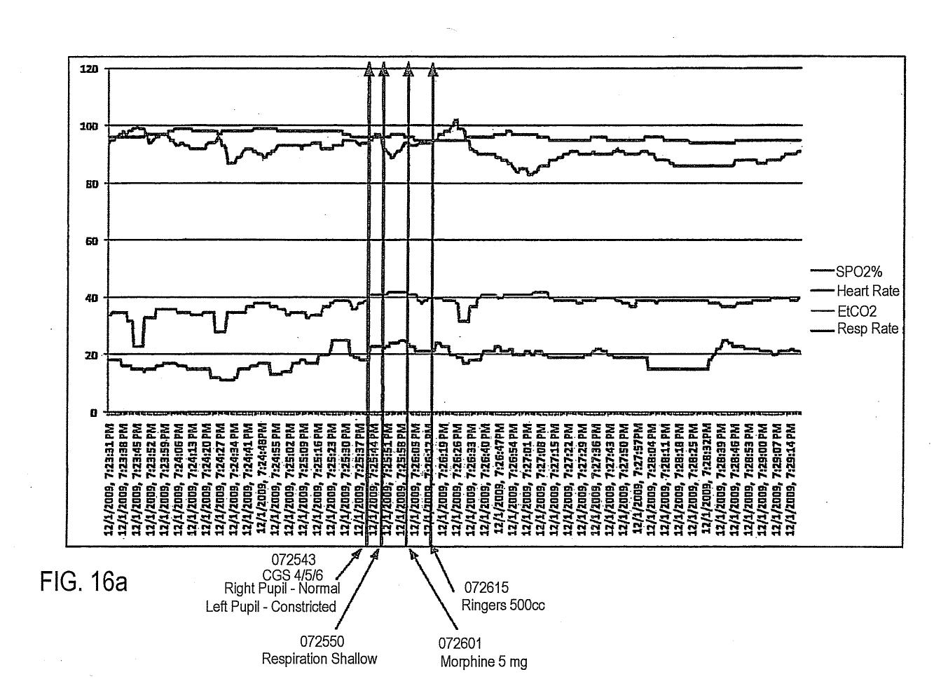

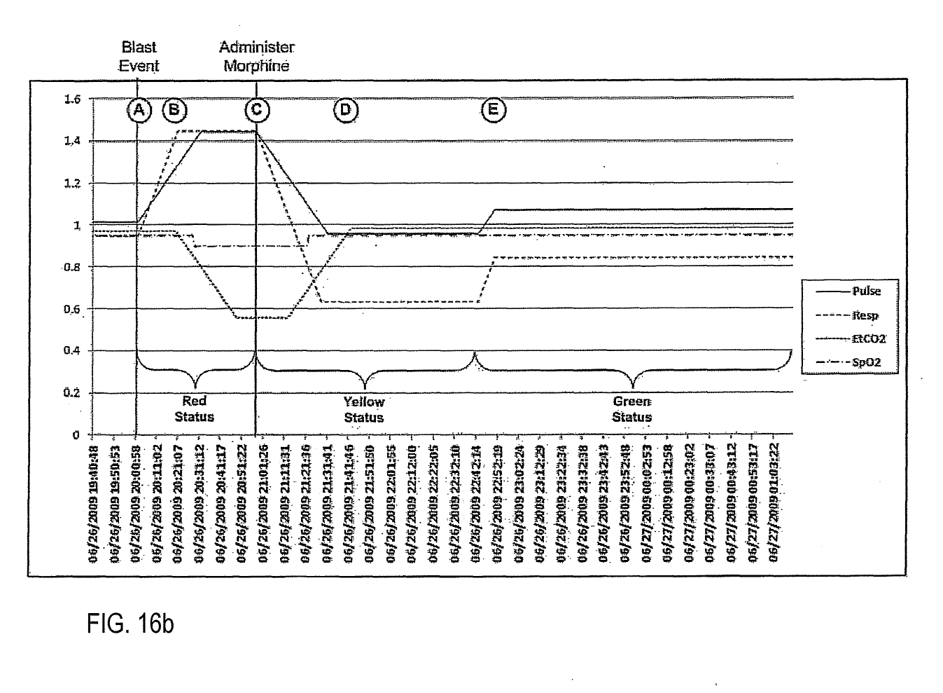

The PTRP will negotiate for “High Power” mode from the host PC but will be tolerant of “Low Power” mode in the event that the host PC refuses the request. The PTRP will also populate and print a standard patient/casualty card, as shown in FIG. 15. The PTRP also coordinates the generation of the vital sign, medication and observation summary plot on a single screen synchronized to the actual date and time of each event, as shown in FIGS. 16(a) and (b). In one embodiment, PTRP 1 supports automatic download of patient vital sign information, such as End Tidal Carbon Dioxide (EtCO₂), Respiration Rate, Saturation of Peripheral Oxygen (SpO₂) and Heart Rate, plus any medications and observations recorded in the field to a field hospital PC. EtCO₂ stands for End Tidal Carbon Dioxide, and is a measurement of the exhaled carbon dioxide, indicating the capability of the patient's respiration system to exchange adequate gasses (e.g., O₂ and CO₂) to sustain life. SpO₂ stands for Saturation of Peripheral Oxygen, and is a measurement of the amount of oxygen being carried in the blood stream indicating the capability of the patient's circulatory system to distribute oxygen throughout the body.

The PTRP 1 includes an “In Service” mode which can be invoked by the “unlock” mechanism used to upgrade memory. While in this configuration, PTRP 1 exposes a number of low-level capabilities for interfacing to external devices (e.g. a PC) for uploading and downloading diagnostic information. The “In Service” mode uses less than 0.01% of the processing power of the CPU when PTRP 1 is not in the “In Service” mode.

The PTRP application level software is upgradeable by the PC pushing software code to be updated into PTRP memory 75 over connection port 40. The PTRP 1 runs an integrity check of the data sent by the PC via a check-sum. The PTRP 1 then replaces the existing software code with the new software code and runs the new code via a self-generated PTRP system reset to enter the “Operational” state. Upon boot from this reset, PTRP 1 is “clean” of all patient data in memory.

The PTRP 1 can also log additional data into the PTRP memory 75 including:
On time (display, pump, charging, etc.);
Feature usage (e.g. frequency of entering or exiting various states);
Use Cases (e.g. number of uploads, number of software upgrades, etc.);
Patient physiological trend indicator status (number of transitions, time spent at each level);
Physiological sensor system status (delay from “On” to valid sensor data);
Continuous first sensor subsystem waveform (e.g., plethysmograph waveform);
Continuous second sensor subsystem waveform (e.g., capnograph waveform);
Ambient temperature; and
Barometric pressure.

The above data fields may be accessible to a PC host over connection port 40 and/or RF transceiver 85 wireless link using dedicated tools that are not distributed to the end user (e.g., first responder/EMT or medical facility personnel).

### Corpsman/Medic Medical Assistant Control Unit (CMMACU)

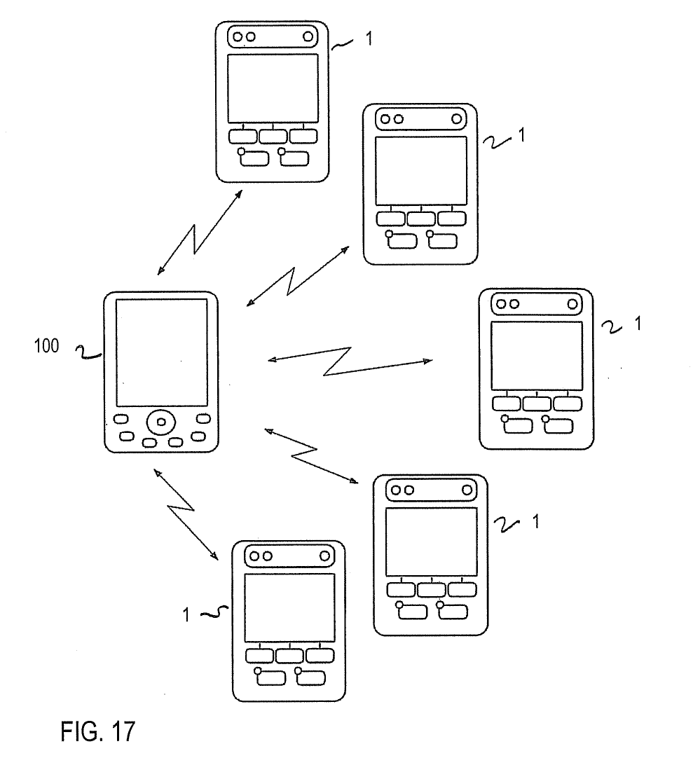

In addition, one or more PTRPs 1 can communicate with a Corpsman/Medic Medical Assistant control unit (CMMACU) 100 that is used by first responders/EMTs in an integrated Corpsman/Medic Medical Assistant (CMMA) system, as shown in FIG. 17. In one embodiment, CMMACU 100 is a commercial off the shelf (COTS) PDA running a software application developed by Sensis that enables CMMACU 100 to interface, transfer data and display data from PTRP 1.

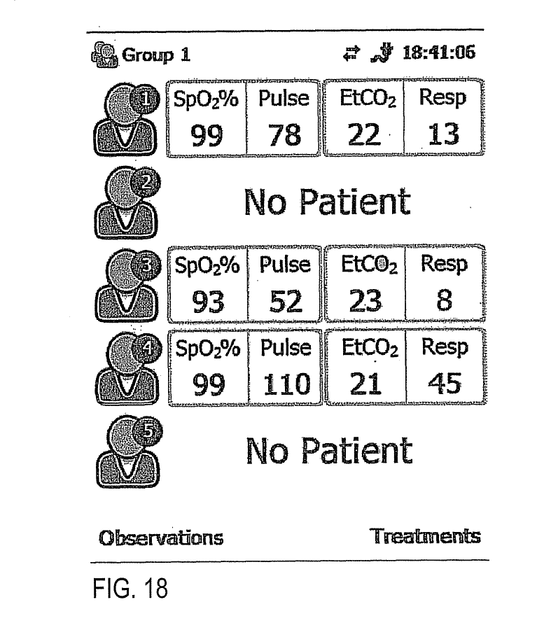

In the CMMA, each CMMACU 100 can interface with and display data from up to five (5) PTRPs 1 on a single display screen. The software application of CMMACU 100 comprises at least the following major components:
a wireless interface communications software program, such as Bluetooth or Ultra-Wide Band, that enables CMMACU 100 to wirelessly connect and communicate to one or more PTPRs 1 in the CMMA system, and
a patient tracking screen that enables the first responder/EMT to simultaneously spot check the vital signs of up to five (5) PTRPs 1 that have been linked to CMMACU 100 on a single display screen as shown in FIG. 18. The CMMACU 100 can be connected and communicating with up to twenty five (25) PTRPs that are grouped by the first responder/EMT into five (5) groups of five (5) PTRPs 1.

For example, in one embodiment of the CMMA system, each PTRP 1 and CMMACU 100 includes an RF transceiver 85 that is a Class II FCC approved Bluetooth radio interface that connects PTRP 1 to and exchanges data with CMMACU 100 without any user action on PTRP 1 or CMMACU 100. The range of the RF Bluetooth transceiver is a minimum of 2 meters and the RF transceiver 85 in each PTRP 1 supports simultaneous connections to up to 5 CMMACUs. The RF Bluetooth transceiver will not fail under the stress condition of “fringe” signal strength where a radio source is cyclically establishing and dropping connection.

The PTRP display 20 displays an RF link status indicator as an icon and PTRP 1 is capable of unambiguously distinguishing the following connection states:

Radio enabled but not connected;

Radio connected to at least one CMMACU; or

Radio Disabled.

The control, authentication, connection, data communication, error detection and correction, as well as the linking or association of one or more PTRPs 1 to CMMACU 100, is performed completely automatically and is transparent to the first responder/EMT. Upon initialization of PTRP 1 after removal from its packaging, PTRP RF transceiver 85 becomes active and begins seeking other wireless devices in the local area, including CMMACUs 100.

After PTRP 1 detects CMMACU 100, PTRP 1 authenticates CMMACU 100 before transmitting any data to CMMACU 100. In one embodiment, PTRP 1 authenticates CMMACU 100 by transmitting a series of digitally encoded data streams that were placed within the communication protocol placed at the time of manufacturing. Only after PTRP 1 successfully authenticates CMMACU 100 will PTRP 1 associate or link with CMMACU 100 and begin transmitting patient vital sign data to the CMMACU.

The PTRP 1 supports data inputs and outputs via the RF transceiver 85 link to CMMACU 100. The PTRP 1 can also receive observations, treatments and patient personal identification information from CMMACU 100 via the RF Bluetooth link and enters that information into the PTRP data log in PTRP memory 75. The PTRP 1 transmits vital sign information from the physiological parameter sensor subsystems 50 and 60, and physiological trend status data to CMMACU 100.

In the case of multiple CMMACUs, PTRP 1 ensures that all information in the data log from the first CMMACU 100 is transmitted (e.g., pushed) to all other CMMACUs, thereby, supporting a first CMMACU 100 transmitting observations while a second CMMACU 100 viewing the observations. In the case of multiple CMMACUs editing and transmitting data simultaneously to PTRP 1, PTRP 1 will accept and acknowledge all receiving inputs while not corrupting the data in the PTRP data log in PTRP memory 75, with the most recent received data inputs being stored in the data log and broadcast to the other CMMACUs. In this sense, CMMACU 100 is considered “stateless” and PTRP 1 performs all calculations and stores all of the data. CMMACU 100 is only a “thin client” display and cannot display different patient data and status than is shown on PRTP 1. If CMMACU 100 loses its wireless connection to PTRP 1 and then re-establishes the connection, PTRP 1 considers this connection as a “new” connection and will not rely on CMMACU 100 to possess any valid information. In this case, PTRP 1 transmits the vital sign data that is currently displayed on PTRP display 20, physiological trend indicator data and notification of transitions between trend status indicators to all connected CMMACUs, including the “new” connection.

On CMMACU 100, the patient vital sign spot checking screen allows the first responder/EMT to simultaneously display (e.g., spot check) the vital signs of up to five (5) patients being monitored by PTRPs 1 that have linked with CMMACU 100. The patient vital sign information, such as SpO₂, pulse rate, EtCO₂ and respiration rate, is displayed as numeric data on the CMMACU primary screen. The CMMACU patient vital sign spot checking screen also displays changes in the background color of the displayed vital sign data, which is based on the trending algorithm range for that vital sign. For example in FIG. 18, the patient identified as WIA-1 on the CMMACU has a respiration rate that is shown to be deteriorating (yellow) in accordance to the vital sign thresholds that are programmed in the trending algorithm of the PTRP. The same color change is shown for the pulse rate of the patient identified as WIA-4.

The software application on the CMMACU includes group identification, as shown in the upper left portion of FIG. 18. The software application enables CMMACU 100 to display the vital sign data for up to five (5) PTRPs simultaneously on the CMMACU patient vital sign spot checking screen and to switch between up to five (5) groups containing up to five (5) PTRPs each, thereby enabling a single CMMACU to monitor up to twenty five (25) patients. The grouping of PTRPs 1 allows the first responder/EMT to group the patient's by the criticality of their injuries for more frequent monitoring of the more severely injured to enhance rapid triage and transport to a medical facility. The grouping of PTRPs 1 also facilitates a “seamless handoff” between individual first responders and EMTs responsible for transporting the patients. A seamless handoff is defined as the point at which the first responder can handoff patient(s) to another first responder/EMT responsible for transporting the patients by simply having the another first responder/EMT change the group identifications of the patient groups on his CMMACU to link to the first responder's group identification for the patients being handed off without the loss of information typically associated with a summarized report and potentially without the first responder talking to the EMT responsible for transporting, for example.

The CMMACU also contains additional capabilities for recording first responder/EMT observation on Observations and Medications sub-screens of the CMMACU. The Observations screen of the CMMACU is used to record information regarding general observations of the patient. However, when the first responder/EMT enters the information and presses “OK” all the relevant information is sent to the appropriate PTRP 1 where it is stored and synchronized with real time when connected to the Hospital PC. No information is permanently stored on CMMACU 100.

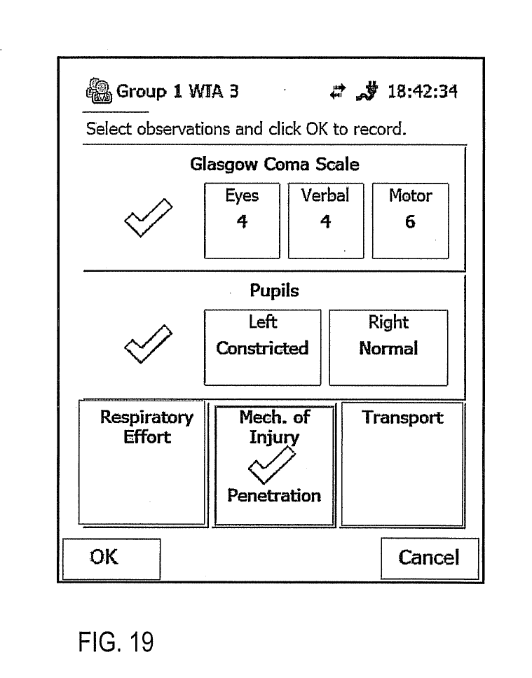

In one embodiment of CMMACU 100, the Observations screen uses the following Glasgow Coma Scale observations, which are displayed to the first responder/EMT, as shown in FIG. 19:

Eyes: numeric scaled values 1-4

Verbal: numeric scaled values 1-5

Motor: numeric scaled values 1-6

Pupils: on a pull down menu
Normal
Constricted
Dilated
No Reaction

Respiratory Effort: on a pull down menu
Shallow
Labored
Labored/Shallow

Mechanism of Injury: on a pull down menu
Crush
Penetration
Blunt
Burn-Chemical
Burn—Thermal
Blast

Transport—on a pull down menu Yes/No

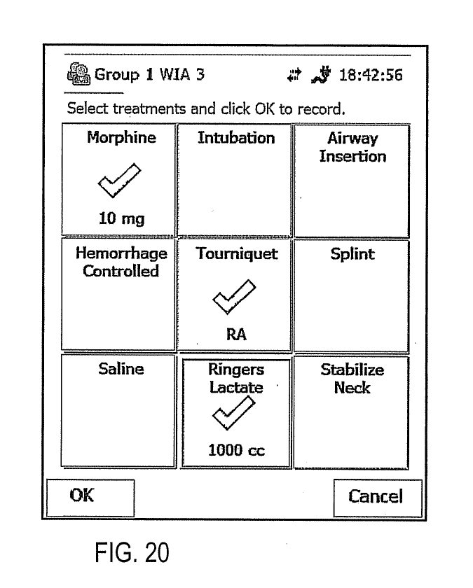

The treatments screen is used to place treatment information into PTRP 1 and, similar to the observations screen, no information is stored on CMMACU 100. After the information is entered, once the “OK” is pressed all of the treatment information is transferred to and stored on PTRP 1. In one embodiment, the following items are displayed on the treatment display screen of the CMMACU to the first responder/EMT, as shown in FIG. 20:
Morphine—5 Mg, 10 Mg, 15 Mg;
Intubation—Yes/No;
Airway Insertion—Nasal, Oral;
Splint—Right Arm, Right Leg, Left Arm, Left Leg;
Tourniquet—Right Arm, Right Leg, Left Arm, Left Leg;
Hemorrhage Controlled—Head, Right Arm, Right Leg, Left Arm, Left Leg, Chest, Abd (abdomen);
Stabilize Neck—Yes/No;
Ringers Lactate—500 cc, 1000 cc, 1500 cc; and
Saline—100 cc, 500 cc, 1000 cc.

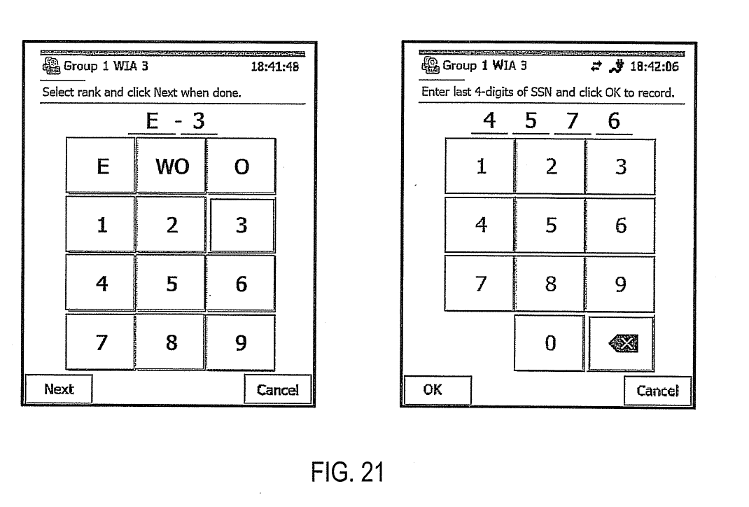

Another set of screens, personal data entry display screens, enables the first responder/EMT to enter personal information related to the patient. The personal information includes name, address, social security number, contact information for next of kin. In one embodiment, the first responder/EMT can enter personal information for military personnel based on military classification (E-Enlisted, WO—Warrant Officer, O—Officer) and rank 1-9 as well as some level of unique for digit code (usually the last four digits of a Serial (Social Security) Number 0-9, as shown in FIG. 21. As with all information entered on CMMACU 100, the personal information that is entered on CMMACU 100 is immediately down loaded and stored in PTRP memory 75 and no data is stored on CMMACU 100.

PTRP First Example

In one example of PTRP 1, PTRP 1 comprises two integral physiological sensors that include sensors and medical hardware and software that are FDA certified. In this example, the PTRP includes the following main components:

A SATCAP Capnography and Pulse Oximetry board;

sensor cables, such as a short nasal cannula and an ear clip;

a CO₂ Pump;

a PTRP controller/display printed circuit board;

an LCD display;

a battery power source;

a PTRP housing; and

PTRP packaging material, including an “OFF switch.

The PTRP 1 and its associated physiological sensor cables 52 and 62 are enclosed in a sealed heat shrunk plastic package. In this embodiment, the sealed packaging includes a label with usage instructions, illustration of attachment to the patient, warnings and approvals (e.g. not tested in accordance with FDA regulations, not tested in accordance with FCC regulations, etc.). The packaging label also provides instructions of how to perform the Ops Check and the expected results of that Ops Check. The sealed packaging also includes a unique identifier which may be a serial number, bar code or both.

In this example, the left event key, (i.e., History event key 21) functions as the “packaged On/Off” switch, which is activated (e.g. “pressed”) when sealed packaging 5 is opened (e.g. a clip). History event key 21 can withstand up to 18 months of continuous depression without releasing (e.g. “un-pressing”) or becoming “stuck.” If History event key 21 becomes stuck due to Mylar deformity or mechanical failure, the PTRP operational mode switch 30 (e.g., Normal/Silent positional switch) acts as a back-up power-on switch for turning the PTRP “On.” The PTRP 1 is still capable of collecting, storing, displaying and transferring physiological data while History event key 21 is stuck in the activated position.

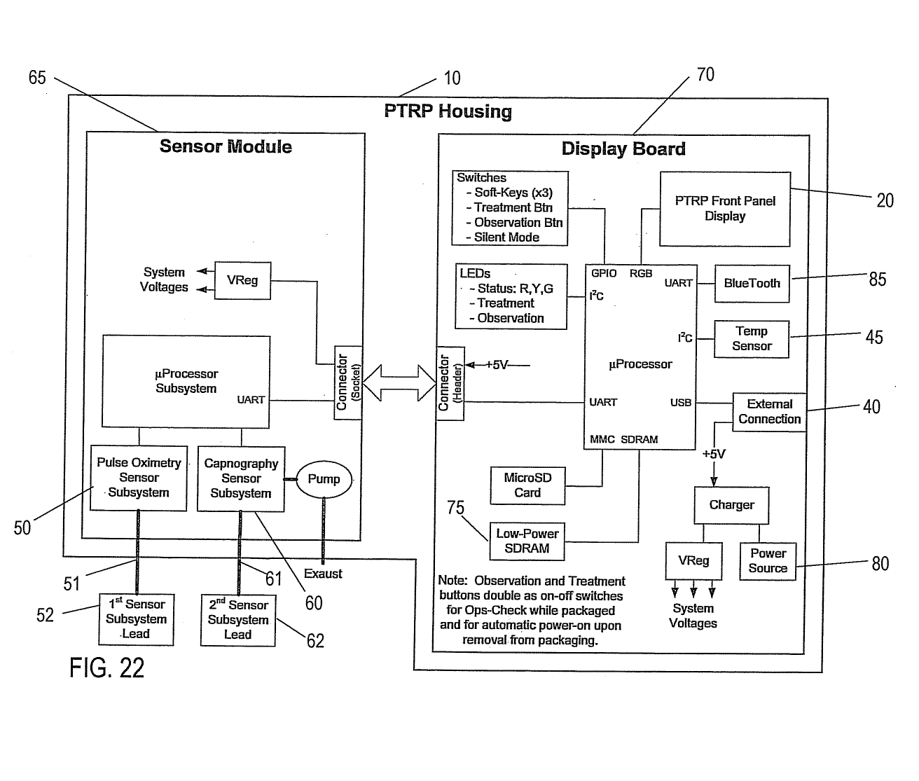

In this example, PTRP 1 is a single enclosed unit that includes two physiological measurements subsystems (i.e., SATCAP Capnography and Pulse Oximetry computational board—specifications for SATCAP Capnography and Pulse Oximetry module manufactured by Treymed, Inc. are hereby incorporated by reference), a PTRP controller/display module 70 containing a single board computer, PTRP memory 75 for data storage, a power source 80 that is contained within PTRP housing 10, PTRP front panel 15 containing three physiological trend indicators 17-19, PTRP display 20 that is capable of presenting numerical physiological sensor data for the integral sensor sub-systems 50 and 60, connection port 40, such as a mini-USB connector, and RF transceiver 85, such as a Bluetooth Radio, as shown in FIG. 22. The PTRP is designed to be carried by the first responders in its packaged configuration. In this example, PTRP 1 weighs a maximum of 12 oz. total weight, including sensors and packaging. The PTRP housing 10 is a maximum of 3.5″ long×2.5″wide×1.5″ height in this example.

In this example, PTRP provides an “Ops Check” capability by simultaneously depressing Observation button 28, which is labeled “Blast”, and Treatment button 26, which is labeled “Morphine” while in the “Packaged” state. In the “Ops Check,” PTRP 1 performs a check of the following subsystems:
Power and power management (Ops Check fails at ¼ power level and below);
PTRP Memory 75 check (including file system);
PTRP display 20 LCD & Backlight;
All LEDS on PTRP Front Panel 15, including physiological trend indicators 17-19 and Observation “Blast” indicator 29 and Treatment “Morphine” indicator 28;
Physiological Sensor Sub-Systems 50 and 60;
RF Transceiver 85; and
All button positions (e.g. “Normal” mode and “stuck at” momentaries).

The PTRP 1 sequentially flashes all 5 LEDs in a round-robin fashion (¼ second on each) which aids the first responder/EMT in noticing a pattern anomaly if one of the LEDs isn't responsive. The PTRP 1 displays a status bar on the LCD of the progress of the “Ops Check” and how close it is to completion. The “Ops Check” is completed in less than 30 seconds and PTRP 1 displays either “Pass” or “Fail”. Upon completion of the Ops Check, PTRP 1 will display the final result for a minimum of 10 seconds and then automatically blank the display and return to the “Packaged” state unless the user continues to invoke the Ops Check after the 10 second time-out (e.g. continues to hold down the “Morphine” and “Blast” buttons). In this case, PTRP 1 continues to display the Ops Check result until the “Morphine” and “Blast” buttons are released. The PTRP 1 ignores all button inputs during the “Ops Check” and does not require any user input to determine pass/fail.

The PTRP 1 can also log more detailed information as to the individual subsystem test results in the data log as necessary for post-mortem failure analysis when the failed PTRPs 1 are returned to a service depot (e.g. battery level, barometric pressure aka. a leak in the packaging, temperature, etc.).

In this example, the first integral physiological sensor subsystem 50 is a capnograph breath gas analyzer capable of measuring and reporting side-stream EtCO2, Capnography and Respiration Rate via first physiological sensor lead 52, a short nasal cannula. The Capnography nasal cannula draws gas (inhalant and exhalent) from the patient's nostrils. The nasal cannula is less than 30 inches in length, preferably 18 inches in length from water trap to the bifurcating connector. Keeping the nasal cannula tube as short as possible minimizes the thermal differential between the patient, ambient environment and PTRP 1. The short length is also advantageous to minimize the amount of resistance required for gas flow through the nasal cannula tube, reducing PTRP 1 power consumption and minimize the contaminants draw into the nasal cannula tube. The nasal cannula attaches to PTRP 1 via a water trap which is seated into a first physiological sensor connection port 51 that is integral to the PTRP housing 10. The first physiological sensor connection port 51 provides a leak-free connection between a water trap that interfaces with first physiological sensor connection port 51 in this embodiment and the pneumatic tubing inside PTRP housing 10. The water trap and nasal cannula can be replaced in the field by first responders/EMTs.

In this example, the second integral physiological sensor is a pulse oximeter hemoglobin configuration analyzer capable of measuring and reporting SpO2, plethysmography, and Heart Rate via a reusable ear clip, other disposable SpO2 sensor or pediatric SpO2 sensor, as needed. The pulse oximetry cable connects to PTRP 1 in a sealed, strain relieved second physiological sensor connection port 61 on PTRP 1. The pulse oximetry cable contains analog and digital signals to drive LEDs and detectors across any physiologically relevant feature on the patient (e.g. ear, finger, etc.). The pulse oximetry cable is 30″ long when measured from the second physiological sensor connection port 61 on PTRP housing 10 to the tip of the ear clip. The length for the pulse Oximetry cable was selected to accommodate PTRP 1 being positioned at the patient's head while stretching comfortably to the patient's finger. Typical usage would be the cable stretching from PTRP 1 on the patient's chest to patient's ear.

The printed circuit board (PCB) providing the physiological parameters is the TreyMed SatCap Capnography and Pulse Oximetry PCB. The PTRP 1 also includes a custom controller/display PCB that interfaces with the SatCap PCB. The custom controller/display PCB provides the communication path between the SatCap physiological data and PTRP display 20, CMMACU 100 and hospital PC, for example.

In this example, the PTRP's internal non-volatile memory 75 can store the following data:
Heart Rate every 1 Hz;
Respiration Rate every 1 Hz;
EtCO₂ every 1 Hz;
SpO₂ every 1 Hz;
20 Observations;
20 Treatments;
36 Ops Check results; and
Patient identification data.

After connecting the PTRP physiological sensor sub-system leads 52 and 62 to PTRP 1 and the patient, the PTRP is placed on the patient's chest, placed on the ground beside the patient, or strapped to the gurney or stretcher on which the patient is lying. The PTRP 1 is connected to the patient, gurney or stretcher using double sided adhesive tape attached to the back surface 11 of the PTRP housing 10. The PTRP 1 will collect, record and display patient vital sign data and physiological trend status. The data collection starts automatically after power-on (e.g. removal from package or user invoked) and PTRP 1 continues to log data and status until it enters the “Hibernate” state due to imminent power failure.

In the operational mode, PTRP 1 uses two primary mechanisms to report vital sign data for the patient to the first responder/EMT. The first is the three physiological trend indicators 17-19 and the second is PTRP display 20 on PTRP front panel 15. In this example, the physiological trend indicators comprise a series of three LEDs (left to right in order green physiological trend indicator 17, yellow physiological trend indicator 18, and red physiological trend indicator 19) for trend reporting and a color LCD display 20 is utilized for real-time vital sign real-time display of measured vital sign values. In the operational state, PTRP display 20 is an LCD and PTRP 1 will constantly power the LCD backlight so that the first responder/EMT does not need to physically interact with PTRP 1 just to bring a dimmed backlight back up to full visibility to “glance” at the displayed vital sign data of the patient. The ability of the first responder/EMT to “glance” at PTRP display 20 to determine the patient's status is an important feature of PTRP 1 and PTRP 1 maintains PTRP display 20 active at the expense of extended battery life (i.e., power consumption).

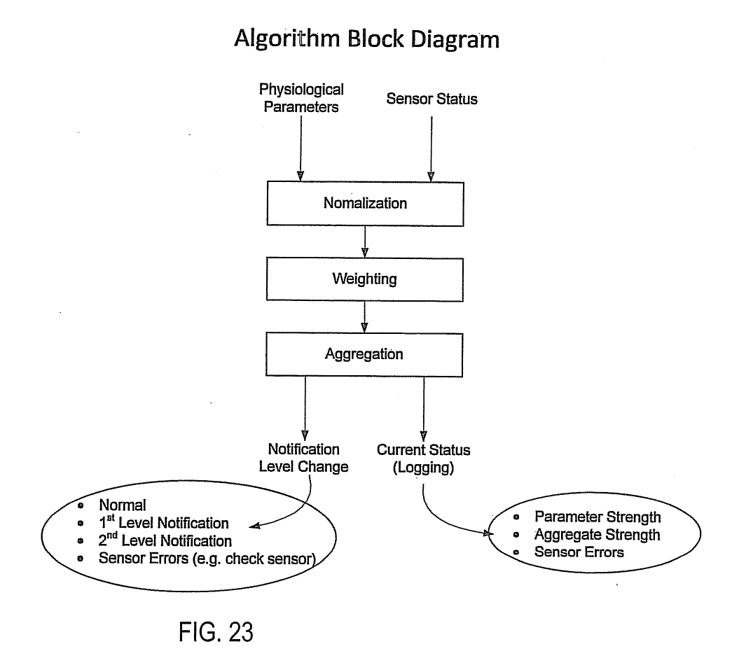

The physiological trend indicators 17-19 report the physiological trend of the patient that is determined by PTRP 1 using a least lower bound algorithm based on the data from the sensor sub-systems 50 and 60 integral to the PTRP. An example of the data flow in the least lower bound algorithm is shown in FIG. 23. In this embodiment, the least lower bound algorithm normalizes data from the first and second sensor sub-systems 50 and 60, weighs the normalized data and aggregates the weighted data to determine the applicable physiological status for each displayed parameter, as shown in FIG. 23. In this example, the algorithm detects sensor sub-systems 50 and 60 faults and does not use invalid data based on that sensor fault to influence the physiological trend indicators 17-19 or be displayed on PTRP display 20. Often times these faults are associated with the sensor leads 52 and 62 (EtCO₂ or SpO₂) falling off the patient, misplaced, crimped or crushed. In these instances PTRP display 20 will display a single fault indication or a double fault indication. No special tools are needed and no software diagnostics suggestions are provided to the first responder/EMT.

The results of the least lower bound algorithm coupled with a standard list of vital sign thresholds is used to illuminate physiological trend indicators 17-19 to show the current status and trend information of the patient's condition. In this embodiment, the following are the thresholds for determining the behavior of physiological trend indicators 17-19 and displayed numerical data on PTRP 1:

TABLE 2
Physiological Parameter Values for LED Thresholds

| | 2^{nd} Level | 1^{st} Level | | 1^{st} Level | 2^{nd} Level |
|---|---|---|---|---|---|
| | Notification | Notification | Normal | Notification | Notification |
| | (Red) | (Yellow) | (Green) | (Yellow) | (Red) |
| Pulse Rate | PR < 40 | 40 ≦ PR < 55 | 55 ≦ PR ≦ 90 | 90 < PR ≦ 100 | PR > 100 |
| Resp Rate | RR < 8 | 8 ≦ RR < 13 | 13 ≦ RR ≦ 22 | 22 < RR ≦ 26 | RR > 26 |
| % SpO₂ | SpO₂ < 90 | 90 ≦ SpO₂ < 95 | 95 ≦ SpO₂ ≦ 100 | N/A | N/A |
| EtCO₂ | EtCO₂ < 25 | 25 ≦ EtCO₂ < 30 | 30 ≦ EtCO₂ ≦ 43 | 43 < EtCO₂ ≦ 47 | EtCO₂ > 47 |

The threshold values are programmed into PTRP 1 and can be changed to different values based on the installed physiological sensors, patient needs (e.g., pediatric patient, COPD patient etc.) or in accordance with the latest medical data by updating the PTRP software. In this example, when the patient's status is changing from one state (e.g., green) to another (e.g., yellow), the physiological trend indicators 17-19 indicate the trend of the patient's status by showing the current state as a solid light (e.g., green) and the new state (e.g., yellow) as a blinking light at a 1 Hz, 25% duty cycle. The PTRP 1 displays this transition state for 10 seconds before the patient's new current state is displayed as a solid light (e.g., yellow) and the previous state (e.g., green) as off. The PTRP 1 provides hysteresis of 30 seconds before displaying another status transition in order to minimize the flashing lights on a patient who's hovering in the threshold between two states.

The algorithm requires two inputs:
1) physiological data from the physiological sensor subsystems, and
2) status of the physiological sensor subsystems (e.g. tube clogged, no patient found, etc.)
   The algorithm generates two outputs:
1) a change notification for the status level (including fault status for one or both of the physiological sensor subsystems), and
2) a stream of the most recently derived status data.

Since the physiological sensor subsystems measure physiological data having varying types of data and data range values, the physiological sensor data is normalized before the data is weighted and aggregated. The basic normalization method is a distance function where the greater the distance away from the ideal value range for that measured physiological parameter the greater the strength of the measured parameter. In this embodiment, each of the measured physiological parameters has an ideal value which is roughly the center point of the “Normal” region as shown in Table 3:

TABLE 3
Optimum Physiological Parameter

| HR | SpO₂ | RR | EtCO₂ |
|---|---|---|---|
| 70 | 100 | 19 | 35 |

For the parameters of heart rate (HR), respiratory rate (RR) and EtCO2, the measured value can either decrease or increase, such that the patient status thresholds are either above or below the optimum values shown in Table 3. For algorithmic simplification, in this embodiment the values for these parameters are measured as decreasing with only two thresholds that symmetrically represent both higher and lower values. The first normalization step for these parameters is shown below:

| Literals: | |
|---|---|
| #DEFINE HR_OPTIMUM | 70 |
| #DEFINE RR_OPTMUM | 19 |
| #DEFINE SPO2_OPTIMUM | 100 |
| #DEFINE ETCO2_OPTIMUM | 35 |

Logic:
HRdistance = abs(HR_OPTIMUM − HRmeasured)
RRdistance = abs(RR_OPTIMUM − RRmeasured)
EtCO2distance = abs(ETCO2_OPTMUM − EtCO2measured)
SpO2distance = SPO2_OPTTIMUM − SpO2measured

The next normalization step is to calculate the strength of each measured physiological parameter for direct comparison. The measured physiological parameters are normalized according to their thresholds, and the thresholds are set according to their margin away from the optimum value. In this embodiment, the margin is determined by percentage.

After the strength of each of the measured physiological parameters is determined, the measured physiological parameters are weighted. The weighting factor is used to give priority to the parameters that are “more important” in the determination of the physiological trend status of the patient, as shown in Table 4.

TABLE 4
Percentages for Notification Thresholds

| | HR | SpO₂ | RR | EtCO₂ |
|---|---|---|---|---|
| Optimum Health Point | 70 | 100 | 19 | 35 |
| 1^{st} Level Notification | 25% | 5% | 25% | 17% |
| 2^{nd} Level Notification | 40% | 10% | 50% | 30% |
| Weighting Factor | 1 | 0.9 | 0.6 | 0.6 |

The first step in weighting is to assign the “importance” of each parameter. As shown below, the weightings for the four parameters in this embodiment are defined as:

### Literals

| | |
|---|---|
| #DEFINE HR_WEIGHTING | 1 |
| #DEFINE SPO2_WEIGHTING | 0.9 |
| #DEFINE RR_WEIGHTING | 0.6 |
| #DEFINE ETCO2_WEIGHTING | 0.6 |

The weighting will be symmetric around the zero point which is the threshold between Green (e.g. “Normal”) and Yellow (e.g. Alert). Thus any application of weighting will not affect that threshold. However, since there are two levels of notification: Yellow vs. Red; the Red threshold has to be adjusted according to the weighting, both individually and aggregate.

After the single instance weighting, the historical weighting must be taken into account. This part of the algorithm supports a condition where one of the individual parameters is signaling a notification change but the total weight of the parameters is not. If this condition persists, the algorithm will eventually signal a change of the notification level.

In this embodiment, the Heart Rate measured data is the most heavily weighted measurement because it is derived by the Pulse Oximetry sensor. The SpO2 measurement is a latent physiological parameter compared to heart rate so it is weighted slightly lower. The respiration rate is derived from the CO2 measurements from the patient's nasal cannula and is susceptible to variation based on normal patient behaviors such as talking, sneezing, coughing, etc. Therefore the weighting is lower for CO2 measurements than for Pulse Oximetry measurements.

For example, as illustrated in the table below, the Heart Rate and SpO2 percentage are squarely in the “green” region but the Respiration Rate is “yellow” and even advances to “red” while the EtCO2 remains “yellow”. Given the patient's health is a combination of the parameters, the heart rate and saturation suggest the patient is healthy, but the other two parameters suggest a patient with a concerning condition. Eventually, the patient's heart rate will deteriorate and the strength will decrease. The SpO2 is typically considered a “latent” measurement and will take even more time to change notification levels in a young, healthy trauma patient.

TABLE 5
Parameters with Disparate Notification Levels

| HR | % O₂ | RR | EtCO₂ |
|---|---|---|---|
| 63 | 99 | 24.4 | 28.7 |
| 63 | 99 | 24.4 | 28.7 |
| 63 | 99 | 24.4 | 28.7 |
| 63 | 99 | 24.4 | 28.7 |
| 63 | 99 | 24.4 | 28.7 |
| 63 | 99 | 24.4 | 28.7 |
| 63 | 99 | 24.4 | 28.7 |
| 63 | 99 | 24.4 | 28.7 |
| 63 | 99 | 24.4 | 28.7 |
| 63 | 99 | 24.4 | 28.7 |
| 63 | 99 | 24.4 | 28.7 |
| 63 | 99 | 28.9 | 28.7 |
| 63 | 99 | 28.9 | 28.7 |
| 63 | 99 | 28.9 | 28.7 |
| 63 | 99 | 28.9 | 28.7 |
| 63 | 99 | 28.9 | 29.1 |
| 63 | 99 | 28.9 | 29.1 |
| 63 | 99 | 28.9 | 29.1 |
| 63 | 99 | 28.9 | 29.1 |
| 63 | 99 | 28.9 | 29.1 |
| 63 | 99 | 28.9 | 29.1 |
| 63 | 99 | 28.9 | 29.1 |
| 63 | 99 | 28.9 | 29.1 |
| 63 | 99 | 28.9 | 29.1 |
| 63 | 99 | 28.9 | 29.1 |

In order to support an early notification for the patient illustrated in Table 5, the algorithm supports an increase in strength based on time. A disparate strength is tracked for a fixed number of samples with a sliding window. Further, the strength is adjusted according to the weighting of the parameter. Only in the case where the parameter is disparate with the current state is the strength be adjusted, but the history must be maintained in support of notification changes.

The history buffer has been chosen based on the standard EMS policy of taking a pulse by counting arterial palpitations for 60 seconds and declaring that as the heart rate. The parameters derived from the Pulse Oximeter are based on the 60 second value, and the parameters derived from Capnography are normalized to the equivalent number of breath cycles that would nominally be used to determine heart rate in 60 seconds.

Finally, the bias has a maximum value per measurement. The maximum is based on the concept that 100 is the maximum value away from the current measurement between yellow and green (e.g. yellow changes at “0” and green has a maximum value of “100”). Note that for green vs. red, the maximum difference is theoretically 300 (e.g. min red: −200, max green: 100). Thus the bias is the same for all three parameters and set to 0.01 such that the maximum contribution to strength per measurement is 1 for neighboring regions (e.g. Yellow to Green) and a maximum of 2 for a far region (e.g. Red to Green).

Literals:
#DEFINE HR_HISTORY 60
#DEFINE RR_HISTORY (HR_OPTIMUM / RR_OPTIMUM) * 60
#DEFINE SPO2_HISTORY 60
#DEFINE ETCO2_HISTORY (HR_OPTIMUM / RR_OPTIMUM) * 60

| | |
|---|---|
| #DEFINE HR_MAX_BIAS | 0.01 |
| #DEFINE RR_MAX_BIAS | 0.01 * (HR_HISTORY / RR_HISTORY) |

#DEFINE SPO2_MAX_BIAS 0.01
#DEFINE ETCO2_MAX_BIAS 0.01 * (HR_HISTORY / ETCO2_HISTORY)
#DEFINE HYST_GREEN 10

| | |
|---|---|
| #DEFINE HYST_YELLOW | 10 |

#DEFINE HYST RED 10
ENUM
{

STATUS_GREEN,
STATUS_YELLOW,
STATUS_RED,
STATUS_ERROR,
STATUS_CURRENT

}
Logic:
/* back out the expired history from the bias */
HRhistoryBias −= HRhistory[HR_HISTORY]
RRhistoryBias −= RRhistory[RR_HISTORY]
SpO2historyBias −= SpO2history[SPO2_HISTORY]
EtCO2historyBias −= EtCO2history[ETCO2_HISTORY]
switch (CurrentCasualtyStatus)
{

case STATUS_GREEN:

if HRstrength <= THRESH_YELLOW

HRhistory[current] = HRstrength * HR_MAX_BIAS

else

HRhistory[current] = 0 /* no strength history this parameter */

if RRstrength <= THRESH_YELLOW

RRhistory[current] = RRstrength * RR_MAX_BIAS

else

RRhistory[current] = 0 /* no strength history this parameter */

if SpO2strength <= THRESH_YELLOW

SpO2history[current] = SpO2strength * SPO2_MAX_BIAS

else

SpO2history[current] = 0 /* no strength history this parameter */

if EtCO2strength <= THRESH_YELLOW

EtCO2history[current] = EtCO2strength * ETCO2_MAX_BIAS

else

EtCO2history[current] = 0 /* no strength history this parameter */

break;

case STATUS_YELLOW:

if (HRstrength > THRESH_YELLOW)

HRhistory[current] = HRstrength * HR_MAX_BIAS

elseif (HRstrength <= THRESH_RED)

HRhistory[current] = (HRstrength − THRESH_RED)*

HR_MAX_BIAS

else

HRhistory[current] = 0 /* no strength history this parameter */

if (RRstrength > THRESH_YELLOW)

RRhistory[current] = RRstrength * RR_MAX_BIAS

elseif (RRstrength <= THRESH_RED)

RRhistory[current] = (RRstrength − THRESH_RED)*

HR_MAX_BIAS

else

RRhistory[current] = 0 /* no strength history this parameter */

if (SpO2strength > THRESH_YELLOW)

SpO2history[current] = SpO2strength * SPO2_MAX_BIAS

elseif (SpO2strength <= THRESH_RED)

SpO2history[current] = (SpO2strength − THRESH_RED)*

HR_MAX_BIAS

else

SpO2history[current] = 0 /* no strength history this parameter */

if (EtCO2strength > THRESH_YELLOW)

EtCO2history[current] = EtCO2strength * ETCO2_MAX_BIAS

elseif (EtCO2strength <= THRESH_RED)

EtCO2history[current] = (EtCO2strength − THRESH_RED)*

HR_MAX_BIAS

else

EtCO2history[current] = 0 /* no strength history this parameter */

break;

case STATUS_RED:

if HRstrength > THRESH_RED

HRhistory[current] = (HRstrength − THRESH_RED) *

HR_MAX_BIAS

else

HRhistory[current] = 0 /* no strength history this parameter */

if RRstrength > THRESH_RED

RRhistory[current] = (RRstrength − THRESH_RED) *

RR_MAX_BIAS

else

RRhistory[current] = 0 /* no strength history this parameter */

if SpO2strength > THRESH_RED

SpO2history[current] = (SpO2strength − THRESH_RED) *

SPO2_MAX_BIAS

else

SpO2history[current] = 0 /* no strength history this parameter */

if EtCO2strength > THRESH_RED

EtCO2history[current] = (EtCO2strength − THRESH_RED) *

ETCO2_MAX_BIAS

else

EtCO2history[current] = 0 /* no strength history this parameter */

break;

}
AggregateThreshRed = 0
AggregateHystGreen = 0
AggregateHystYellow = 0
SkipAggregation = TRUE
if (SpO2SensorError == TRUE)
{

HRstrength = 0
SpO2strength = 0
HRhistory[current] = 0
SpO2history[current] = 0

}
else
{

HRstrength *= HR_WEIGHTING
SpO2strength *= SPO2_WEIGHTING
HRhistory[current] *= HR_WEIGHTING
SpO2history[current] *= SPO2_WEIGHTING
AggregateHystGreen += HYST_GREEN * (HR_WEIGHTING +

SPO2_WEIGHTING)

AggregateHystYellow += HYST_YELLOW * (HR_WEIGHTING +

SPO2_WEIGHTING)

AggregateThreshRed += THRESH_RED * (HR_WEIGHTING +

SPO2_WEIGHTING)

SkipAggregation = FALSE;

}
if (CO2SensorError == TRUE)
{

RRstrength = 0
EtCO2strength = 0
RRhistory[current] = 0
EtCO2history[current] = 0

}
else
{

RRstrength *= RR_WEIGHTING
EtCO2strength *= ETCO2_WEIGHTING
RRhistory[current] *= RR_WEIGHTING
EtCO2history[current] *= ETCO2_WEIGHTING
AggregateHystGreen += HYST_GREEN * (RR_WEIGHTING +

ETCO2_WEIGHTING)

AggregateHystYellow += HYST_YELLOW * (RR_WEIGHTING +

ETCO2_WEIGHTING)

AggregateThreshRed += THRESH_RED * (RR_WEIGHTING +

ETCO2_WEIGHTING)

SkipAggregation = FALSE;

}
/* Adjust current bias with new input */
HRhistoryBias += HRhistory[current]
RRhistoryBias += RRhistory[current]
SpO2historyBias += SpO2history[current]
EtCO2historyBias += EtCO2history[current]
/* note that “history” is likely to be a circular buffer or a linked list and a
simple increment is only illustrative of the concept of sliding the history
window by one */current++

The bias will incrementally accumulate non-zero values in the buffer for measured physiological parameters that do not support the current displayed physiological trend indicator displayed by PTRP 1, and incrementally accumulate zero values when the measured physiological parameters support or agree with the current displayed physiological trend indicator. In logical terms, the bias smoothly increases or decreases when the notification level changes. In the present invention, a single, anomalous “spike” is averaged in terms of bias across the entire history to prevent random changes in the displayed physiological trend indicator for the patient.

The next step in normalization is line fitting for each parameter. In this embodiment, the parameter is fit to a line normalized at two points:
(1^{st} Level Notification,0); (2^{nd} Level Notification,−100)
   Given the above two points, the “Green” range is positive, the “Yellow” range is from 0 to −100 and the “Red” range is less than −100, as shown in Table 6.

TABLE 6
Values Used for Line Fitting

| | Optimum | 1^{st} | 2^{nd} | Y | |
|---|---|---|---|---|---|
| | Health Point | Threshold | Threshold | Intercept | Slope |
| HR | 70.00 | 52.50 | 42.00 | 0 | 9.52 |
| HR_Y Values | 166.67 | 0.00 | −100.00 | −500.00 | — |
| SpO₂ | 100.00 | 95.00 | 90.00 | 0.00 | 20.00 |
| SpO_{2—}Y | 100.00 | 0.00 | −100.00 | −1900.00 | — |
| Values | | | | | |
| RR | 19.00 | 14.25 | 9.50 | 0.00 | 21.05 |
| RR_Y Values | 100.00 | 0.00 | −100.00 | −300.00 | — |
| EtCO₂ | 35.00 | 29.05 | 24.50 | 0.00 | 21.98 |
| EtCO_{2—}Y | 130.77 | 0.00 | −100.00 | −638.46 | — |
| Values | | | | | |

The final step is to normalize the “Green” and “Red” regions. The “Green” region extents from the 1^{St} Level Notification point (Y value of 0) to the optimum point, which is different for each parameter. Likewise, the “Red” region extends all the way to the Y Intercept. Thus the “Yellow” region was normalized by definition and the “Red” and “Green” regions must be normalized mathematically. For some of the parameters, the “Green” and “Red” regions are not the same size, and therefore would potentially contribute different strengths for equivalent changes in value. To equalize the strength of these two regions, the longer of the two is truncated to be the same maximum distance as the shorter of the two, as shown below.

Literals:

| | |
|---|---|
| #DEFINE HR_1ST_PERCENTAGE | 0.25 |
| #DEFINE HR_2ND_PERCENTAGE | 0.40 |
| #DEFINE RR_1ST_PERCENTAGE | 0.25 |
| #DEFINE RR_2ND_PERCENTAGE | 0.50 |

#DEFINE SPO2_1ST_PERCENTAGE 0.05
#DEFINE SPO2_2ND_PERCENTAGE 0.10
#DEFINE ETCO2_1ST_PERCENTAGE 0.17
#DEFINE ETCO2_2ND_PERCENTAGE 0.30
/* derived normalization values */
#DEFINE HR_1ST_DISTANCE HR_OPTIMUM *
HR_1ST_PERCENTAGE

| | |
|---|---|
| #DEFINE HR_1ST_THRESHOLD | (HR_OPTIMUM − |

HR_1ST_DISTANCE)
#DEFINE HR_2ND_DISTANCE HR_OPTIMUM *
HR_2ND_PERCENTAGE

| | |
|---|---|
| #DEFINE HR_2ND_THRESHOLD | (HR_OPTIMUM − |

HR_2ND_DISTANCE)

| | |
|---|---|
| #DEFINE HR_MAX_DISTANCE | HR_OPTIMUM * |

(HR_1ST_PERCENTAGE + HR_2ND_PERCENTAGE)
#DEFINE HR_SLOPE (−100−0)/(HR_2ND_THRESHOLD−
HR_1ST_THRESHOLD)

| | |
|---|---|
| #DEFINE HR_B | −HR_SLOPE * HR_1ST_THRESHOLD |

#DEFINE HR_NORM 100 / (HR_SLOPE * HR_OPTIMUM + HR_B)
#DEFINE RR_1ST_DISTANCE RR_OPTIMUM *
RR_1ST_PERCENTAGE
#DEFINE HR_1ST_THRESHOLD (RR_OPTIMUM −
RR_1ST_DISTANCE)
#DEFINE RR_2ND_DISTANCE RR_OPTIMUM *
RR_2ND_PERCENTAGE
#DEFINE RR_2ND_THRESHOLD (RR_OPTIMUM −
RR_2ND_DISTANCE)
#DEFINE RR_MAX_DISTANCE RR_OPTIMUM *
(RR_1ST_PERCENTAGE + RR_2ND_PERCENTAGE)
#DEFINE RR_SLOPE (−100−0)/(RR_2ND_THRESHOLD−
RR_1ST_THRESHOLD)
#DEFINE RR_B −RR_SLOPE * RR_1ST_THRESHOLD
#DEFINE RR_NORM 100 / (RR_SLOPE * RR_OPTIMUM + RR_B)

| | |
|---|---|
| #DEFINE SPO2_1ST_DISTANCE | SPO2_OPTIMUM * |

SPO2_1ST_PERCENTAGE
#DEFINE SPO2_1ST_THRESHOLD (SPO2_OPTIMUM −
SPO2_1ST_DISTANCE)

| | |
|---|---|
| #DEFINE SPO2_2ND_DISTANCE | SPO2_OPTIMUM * |

SPO2_2ND_PERCENTAGE
#DEFINE SPO2_2ND_THRESHOLD (SPO2_OPTIMUM −
SPO2_2ND_DISTANCE)

| | |
|---|---|
| #DEFINE SPO2_MAX_DISTANCE | SPO2_OPTIMUM * |

(SPO2_1ST_PERCENTAGE + SPO2_2ND_PERCENTAGE)
#DEFINE SPO2_SLOPE (−100−0)/(SPO2_2ND_THRESHOLD−
SPO2_1ST_THRESHOLD)
#DEFINE SPO2_B −SPO2_SLOPE * SPO2_1ST_THRESHOLD
#DEFINE SPO2_NORM 100 / (SPO2_SLOPE * SPO2_OPTIMUM +
SPO2_B)

| | |
|---|---|
| #DEFINE ETCO2_1ST_DISTANCE | ETCO2_OPTIMUM * |

ETCO2_1ST_PERCENTAGE
#DEFINE ETCO2_1ST_THRESHOLD (ETCO2_OPTIMUM −
ETCO2_1ST_DISTANCE)
#DEFINE ETCO2_2ND_DISTANCE ECTO2_OPTIMUM *
ETCO2_2ND_PERCENTAGE
#DEFINE ETCO2_2ND_THRESHOLD (ETCO2_OPTIMUM −
ETCO2_2ND_DISTANCE)
#DEFINE ETCO2_MAX_DISTANCE
ETCO2_OPTIMUM*(ETCO2_1ST_PERCENTAGE+ETCO2_2ND_PERCEN
TAGE)
#DEFINE ETCO2_SLOPE (−100−0)/(ETCO2_2ND_THRESHOLD−
ETCO2_1ST_THRESHOLD)
#DEFINE ETCO2_B −ETCO2_SLOPE * ETCO2_1ST_THRESHOLD
#DEFINE ETCO2_NORM 100 / (ETCO2_SLOPE * ETCO2_OPTIMUM +
ETCO2_B)

| | |
|---|---|
| #DEFINE THRESH_YELLOW | 0 |
| #DEFINE THRESH_RED | −100 |

Logic:
if (HRdistance > HR_MAX_DISTANCE)

HRdistance = HR_MAX_DISTANCE

HRstrength = HR_SLOPE * (HR_OPTIMUM − HRdistance) + HR_B
if (HRstrength) > THRESH_YELLOW

HRstrength *= HR_NORM

elseif (HRstrength < THRESH_RED)

HRstrength = ((HRstrength − THRESH_RED) * HR_NORM) +

THRESH_RED
if (RRdistance > RR_MAX_DISTANCE)

RRdistance = RR_MAX_DISTANCE

RRstrength = RR_SLOPE * (RR_OPTIMUM − RRdistance) + RR_B
if (RRstrength) > THRESH_YELLOW

RRstrength *= RR_NORM

elseif (HRstrength < THRESH_RED)

RRstrength = ((RRstrength − THRESH_RED) * RR_NORM) +

THRESH_RED
if (SpO2distance > SPO2_MAX_DISTANCE)

SpO2distance = SPO2_MAX_DISTANCE

SpO2strength = SPO2_SLOPE * (SPO2_OPTIMUM − SpO2distance) +
SPO2_B
if (SpO2strength) > THRESH_YELLOW

SpO2strength *= SPO2_NORM

elseif (SpO2strength < THRESH_RED)

SpO2strength = ((SpO2strength − THRESH_RED) * SPO2_NORM) +

THRESH_RED
If (ETCO2distance > ETCO2_MAX_DISTANCE)

ETCO2distance = ETCO2_MAX_DISTANCE

EtCO2strength = ETCO2_SLOPE * (ETCO2_OPTIMUM − EtCO2distance) +
ETCO2_B
if (EtCO2strength) > THRESH_YELLOW

EtCO2strength *= ETCO2_NORM

elseif (EtCO2strength < THRESH_RED)

EtCO2strength = ((EtCO2strength − THRESH_RED) * ETCO2_NORM) +

THRESH_RED

This achieves symmetry to the algorithm that will limit changes in the “Green” region to have the equivalent effect as changes in the “Red” region.

The next step in the algorithm is the aggregation of the weighted normalized measured physiological parameters into a single physiological trend status for the patient. The core of the logic in the aggregation step is a direct addition of the calculated strengths and then a comparison of this value against the thresholds. This step also enforces some smoothing by maintaining a contiguous count of threshold changes. This is to avoid a “spike” in the data causing a transient change in status. To further smooth the changes, the aggregation step also enforces a hysteresis such that once a 1^{St} or 2^{nd} level notification has occurred; it is “harder” to change to a lower notification level. The hysteresis is defined as 10% of the normalized range.

Literals:
#DEFINE STATUS_CHANGE_COUNT 3
Logic:
AggregateCasualtyStatus = (HRstrength + HRhistoryBias)

+ (RRstrength + RRhistoryBias)
+ (SpO2strength + SpO2historyBias)
+ (EtCO2strength + EtCO2historyBias)

NewCasualtyStatus = STATUS_CURRENT /* default to “no change” */
switch (CurrentCasualtyStatus)
{

case: STATUS_GREEN

if (SkipAggregation == TRUE)

NewCasualtyStatus = STATUS_ERROR

elseif (AggregateCasualtyStatus <= THRESH_YELLOW)
{

StatusYellowCount++
if ((StatusYellowCount) >= STATUS_CHANGE_COUNT)

NewCasualtyStatus = STATUS_YELLOW

}
else

StatusYellowCount = 0 /* not contiguous, clear out and start over */

break

case: STATUS_YELLOW

if (SkipAggregation == TRUE)

NewCasualtyStatus = STATUS_ERROR

elseif (AggregateCasualtyStatus > (THRESH_YELLOW

+AggretateHystGreen))

{

StatusGreenCount++
if ((StatusGreenCount) >= STATUS_CHANGE_COUNT)

NewCasualtyStatus = STATUS_GREEN

}

elseif (AggregateCasualtyStatus <= AggregateThreshRed)
{

StatusRedCount++
if ((StatusRedCount) >= STATUS_CHANGE_COUNT)

NewCasualtyStatus = STATUS_RED

}
else

StatusGreenCount = 0 /* not contiguous, clear out and start over */
StatusRedCount = 0 /* not contiguous, clear out and start over */

break

case: STATUS_RED

if (SkipAggregation == TRUE)

NewCasualtyStatus = STATUS_ERROR

elseif (AggregateCasualtyStatus >

(AggregateThreshRed+AggregateHystYellow))

{

StatusYellowCount++
if ((StatusYellowCount) >= STATUS_CHANGE_COUNT)

NewCasualtyStatus = STATUS_YELLOW

}
else

StatusYellowCount = 0 /* not contiguous, clear out and start over */

break

case: STATUS_ERROR

if (SkipAggregation == TRUE)

/* NOP: Error condition persists, no change to status */

elseif (AggregateCasualtyStatus > THRESH_YELLOW)

NewCasualtyStatus = STATUS_GREEN

elseif (AggregateCasualtyStatus > AggregateThreshRed)

NewCasualtyStatus = STATUS_YELLOW

else

NewCasualtyStatus = STATUS_RED

break

}
if (NewCasualtyStatus != STATUS_CURRENT)
{

SendMessage(NewCasualtyStatus)
CurrentCasualtyStatus = NewCasualtyStatus
StatusYellowCount = StatusGreenCount = StatusRedCount = 0

}

In this embodiment, the algorithm also includes exception handling logic. During initialization, which can be viewed as an exception because there isn't enough history for the algorithm to use in the steps discussed above, the algorithm allows the weighting section to build a partial bias and accept the aggregation's first “status change” as valid. Since the aggregation section of the algorithm includes a “smoothing” function, the algorithm generates a “all sensors faulted” error until it can provide its first valid status. In this embodiment, once the first valid status is displayed, the weighting section of the algorithm continues building its history and if the status changes, simply show the new status as the next valid state. In this embodiment, by initializing the history buffer to a unique value that indicates “I'm not valid data” and the bias value to “0” the algorithm will robustly self-start.

Errors have two external sources: as measured physiological data from the physiological sensor subsystems and in the form of extreme values in the data stream. The algorithm of the present invention does not force an error to the display based on extreme data values in anticipation of support for “full arrest” patients where the vital signs have truly collapsed to zero (e.g. no heart rate) but the patient is still receiving treatments that could re-start the vital signs.

However, internal error handling must take into account these extreme values. In the case of a sensor that has fallen off, a PTRP 1 currently indicating the patient status as “green” (i.e. green physiological trend indicator 17 lit) does not change status to “red” (i.e. red physiological trend indicator 19 lit) when the physiological sensor is inadvertently detached from the patient and the data values have dropped to zero. In this case, when one of the physiological sensor sub-systems indicates an error, the weighting section does not use the data from that physiological sensor sub-system to ensure proper history while in the error state. In this embodiment, no matter what state the system is in, the bias will be zero and thus zero will be inserted into the history buffer. Additionally, the aggregate thresholds are adjusted by removing the parameters from the physiological sensor sub-system indicating an error from the aggregation.

For example, if the ear clip becomes dislodged and generates errors, the aggregate red threshold would be adjusted as:

AggregateThreshRed=(THRESH_RED*SPO2_WEIGHTING+THRESH_RED*HR_WEIGHTING)

In this embodiment, if the algorithm generates an internal error (e.g., out of memory) the error is flagged to the system which determines the appropriate recovery strategy. In this embodiment, the algorithm is “restartable” such that in the case of a system error, the algorithm returns to its initialization state and generates double faults until the algorithm can generate its first valid status.

In this embodiment, the CMMA system also detects and notifies the first responder/EMT of an impending tension pneumothorax condition for a trauma victim. A tension pneumothorax condition, which is frequently encountered by first responders/EMTs in emergency situations, such as vehicle accidents, natural disasters, such as earthquakes, and gunshot victims, is difficult for a first responder to diagnose without a chest x-ray. The CMMA system uses the data fusion algorithm to detect the impending tension pneumothorax condition and notify the first responder/EMT of an impending tension pneumothorax condition when the condition can be treated by needle decompression outside of a medical facility.

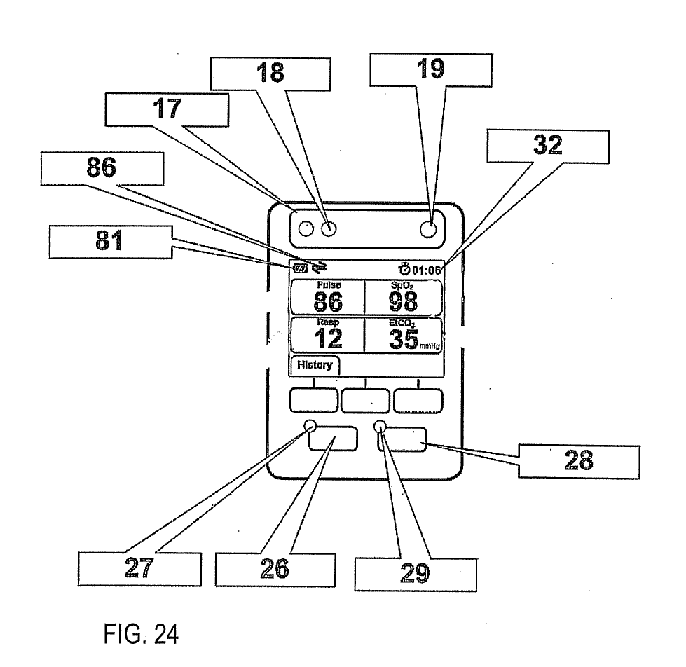

The PTRP display 20 allows direct numeric reading of multiple vital signs being measured by PTRP 1, as shown in FIG. 24. The display screen displays data in the “landscape” orientation and has a pixel array of at least ¼ VGA (e.g. 320×240 pixels). In this example, the four vital signs, EtCO₂ in mmHg, Respiration Rate in breaths per minute, Heart Rate in beats per minute and SpO₂ in % saturated Oxygen, are measured by the integral physiological sensors and updated to the display screen at a 1 Hz rate. All vital sign information displayed on PTRP display 20 is simultaneously stored in PTRP memory 75 at the same 1 Hz data rate. Additionally, PTRP display 20 includes the following indicators:
Elapsed time counter 32 that counts from 00:00 at PTRP initialization. The elapsed time counter 32 can provide real-time date and time information when synchronized with a PC, which provides the real time that medications were administered.
Power level indicator 81, is a Battery life icon that graphically depicts the status of PTRP power source 80 (e.g., battery).
RF link status indicator 86 is an icon that shows whether the RF wireless link is connected or disconnected;
Three event keys are provided on the lower portion of PTRP display 20.

The three event keys in this embodiment are, from left to right, History event key 21, OK event key 22 and CANCEL event key 23.

In this example, History event key 21 displays the patient history of data recorded via Treatment button 26, labeled “Morphine,” and Observation button 28, labeled “Blast,” by the first responder/EMT into PTRP memory 75. In this example, when the “History” event key button on the display is depressed, the display presents a summary screen of the current morphine and blast injury information.

In this example, Treatment button 26 is labeled “Morphine,” and Observation button 28 is labeled “Blast” on PTRP front panel 15, and these buttons provide the first responder/EMT a method for immediate storage of vital observation and medication information.

The Treatment button 26, labeled “Morphine,” enables the first responder/EMT to record administration of morphine or other pain control medication to the patient. Upon pressing of the “Morphine” treatment button 26, the associated “Morphine” indicator 27, Morphine LED illuminates a blue LED and flashes a specific On/Off periodicity relative to the number of doses given to the patient. More specifically, the Morphine LED flashes as follows:

1 dose: ¼ second on, ¾ seconds off;

2 doses: ¼ second on, ¼ second off, ¼ second on, ¾ seconds off; and

3 doses: ¼ on, ¼ off, ¼ on, ¼ off, ¼ on, ¾ off, etc.

The above blinking/off pattern is continued up to a maximum of 5 doses/blinks. If the first responder/EMT presses the “Morphine” treatment button 26 more than 5 times, button presses in excess of 5 will not increase the blink count, but the event will be logged into PTRP memory 75 and PTRP display 20 will display the total count/number of “Morphine” button depressions. In addition, the following three automatic internal operations are performed: first the “Morphine” treatment button 26 depression is correlated with an elapsed time from the elapsed time indicator of PTRP 1, second the button depression is recorded in PTRP memory 75, and third a confirmation screen is displayed on PTRP display 20 that allows the first responder/EMT to confirm or cancel the recording of the pain medication administration. More specifically, when “Morphine” treatment button 26 is depressed, indicating that a dose of pain medication has been administered to the patient, PTRP display 20 presents a confirmation screen to the first responder/EMT that enables the first responder/EMT to either confirm or cancel the recording of the pain medication administration by depressing the “OK” event key or the “CANCEL” event key, respectively, as shown in FIG. 25. If the first responder/EMT fails to depress either the “OK” or “CANCEL” event key within five (5) seconds, the PTRP will automatically confirm and record the pain medication administration. The PTRP records the number of doses and the associated elapsed time of the administration of the medication in memory. Once the “Morphine” treatment and observation button is confirmed, it cannot be undone. PTRP display 20 will display when the last dose of morphine was administered to the patient when the History event key 21 is depressed to assist the users to avoid over medicating the patient. In one embodiment, a information/attention icon is displayed to remind the first responder/EMT that sufficient time has elapsed since the last dose of morphine was administered and another dose of morphine can be administered to the patient, if needed.

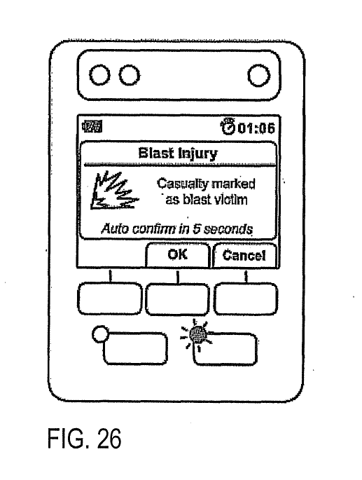

In this example, the Observation button 28 is labeled “BLAST” and enables the first responder/EMT to associate the type of injury suffered by the patient, in particular blunt force trauma from a blast. When the “BLAST” Observation button 28 is depressed by the first responder/EMT, the “BLAST” observation indicator 29, BLAST LED illuminates a blue LED that starts flashing at a 1 Hz rate with a 25% duty cycle and following three automatic internal operations are performed: first “BLAST” Observation button 28 depression is correlated with an elapsed time from the elapsed time indicator of PTRP 1, second “BLAST” Observation button 28 depression is recorded in PTRP memory 75, and third a confirmation screen is displayed on PTRP display 20 that allows the first responder/EMT to confirm or cancel the recording of the first responder/EMT observation that the injury suffered by the patient is a result of a blunt force trauma from a blast, as shown in FIG. 26. If the first responder/EMT fails to depress either the “OK” or “CANCEL” event key within five (5) seconds, PTRP 1 will automatically confirm and record the first responder/EMT observation. Once the “BLAST” Observation button 28 is confirmed, it cannot be undone.

The “BLAST” Observation button 28 and “Morphine” Treatment button 26 and their respective indicators 29 and 27 may be operated and/or changed at CMMACU 100 as well as by pressing the buttons on PTRP front panel 15.

In this example, PTRP 1 supports automatic download of patient vital sign information, EtCO₂, Respiration Rate, SpO₂ and Heart Rate, plus any medications and observations recorded on the PTRP to a hospital PC. Since, within PTRP 1 resides the comprehensive software required to download the information (e.g., MS Excel macros), no pre-loading of software is required for the hospital PC. The PTRP interfaces directly to the hospital PC via connection port 40 and the information is automatically downloaded.

Upon connection to the hospital PC, PTRP 1 synchronizes its on-board elapsed time to the PC's real-time clock to calculate the overall real-time of the PTRP's data since it was turned on and connected to the patient. The PTRP 1 also automatically formats the raw patient vital sign, observation and medication information into an Excel spreadsheet format for printing.

In this example, upon connection to the hospital PC, the PTRP also coordinates automatic filling in of critical patient information to a Patient Casualty Card. This information consists of (at a minimum): Time, day/night incident, fluids, medications, comments (blast), recent pulse, respiration and trend data.

The PTPR resident interface software plots and prints all four physiological parameters (Heart Rate, SPO₂ saturated O₂ percentage, EtCO₂ partial pressure and Respiration Rate) on a single plot as shown in FIG. 16(a). Medication inputs are indicated at the correct date and time (as corrected from elapsed time in the PTRP memory) as a red vertical arrow and observations are indicated at the correct date and time (as corrected from elapsed time in the PTRP memory) as a black vertical arrow, as shown in FIG. 16(a). In this embodiment, the PTRP also supports automatic secure File Transfer Protocol (FTP) between the primary software development platform and the PTRP directly through the PTRP supplied miniature USB connector.

In another embodiment, the PTPR resident interface software plots and prints all four physiological parameters (Heart Rate, SPO₂ saturated O₂ percentage, EtCO₂ partial pressure and Respiration Rate) on a single plot as shown in FIG. 16(b). For example, the patient is injured and a PTRP 1 is connected to the patient at “A” in FIG. 16(b). The patient's vital signs deteriorate due to the onset of compensated shock conditions (e.g. accelerated heart rate, accelerated respiration rate) between “A” and “B” and the Casualty Status Algorithm of the present invention indicates a physiological status of the patient as “Red” during the onset of the shock condition, as annotated on FIG. 16(b). At “C” the EMT or Medic administers morphine to the patient and between C and D the patient's vital signs show the physiological response to the morphine and the physiological status of the patient changes to “Yellow”. At “E” in FIG. 16(b), the pain alleviation effect of the administered morphine begins wearing off and the EMT or Medic has stabilized the patient such that the vital signs return to baseline and the physiological status of the patient is shown as “Green”.

FIG. 16(b) emphasizes the normalized vital sign reporting method where the baseline value for each physiological parameter is normalized to a value of 1. The patient's physiological response to trauma and treatment is thus represented by the vital signs trending away from baseline when the patient's condition is deteriorating and towards the baseline when the patient's condition is improving. This composite graph is also representative of the method of analysis used for the Casualty Status Algorithm in this embodiment of the present invention.

Expected Workflow for the PTRP

1) Initial treatment of patient per standing protocols;

2) Open Package;
a. Rip the top off the package;
b. Remove PTRP from vacuum packaging;
c. Remove protective packaging from PTRP;
d. Observe lights as feedback that device is responsive;
e. Extend sensors cables (SpO₂ & CO₂);

3) Attach Device to patient;
a. Clip SpO₂ sensor to patient's ear;
b. Insert Nasal Cannula into patient's nose (dress behind ears);
c. Place PTRP onto patient's Chest;

4) Observe initial patient physiological trend indicator Status;

5) Enter Observations & Treatments into PTRP;
a. Press “blast” button if patient has suffered IED explosion or equivalent;
b. Press “morphine” button for each dose of morphine/pain medication administered to patient;

6) Transport patient to pick-up point;

7) Transfer patient care to the EMT transport personnel;

8) Load patient onto evacuation vehicle;

9) Transport patient to medical facility;

10) Transfer patient care to the medical facility personnel;

11) Download data from PTRP into Hospital computer;
a. Disconnect PTRP from patient (assumes PC and patient are not within a cable length);
b. Connect PTRP to computer via standard USB cable;
c. Open the Microsoft Excel template file found on the PTRP and print patient vital sign, treatment and observation data in tabular or graphical form and print patient data to patient emergency card; and

12) Dispose of PTRP.

CMMACU Example

In this embodiment, The CMMACU 100 hardware platform is the Amrel DA5-1 PDA, and the CMMACU PDA software platform is based upon the Microsoft Windows CE operating system. The CMMACU 100 operates on standard commercial battery power and is capable of continuous operation for a minimum of 8 hours. The CMMACU 100 is recharged using standard commercial battery chargers and mechanisms. The CMMACU 100 includes a wireless communications subsystem that is compatible with PTRP 1.

The first responder/EMT organizes the patients on CMMACU 100 display screens by associating the PTRP icons on the display screen of CMMACU 100 with the PTRPs 1 attached to the patients. In this embodiment, the mechanism for association of PTRP 1 with CMMACU 100 is selecting the patient's PTRP on CMMACU 100, and when association processing starts, PTRP 1 flashes the LCD backlight at ¼ second on, ¼ second off for the duration of the selection association. The first responder/EMT then confirms the association. This association mechanism allows the first responder/EMT who is organizing his screens to select a patient PTRP, look for the flash and either accept or reject this screen organization. Note that this association mechanism is actually a lower power state on PTRP 1 than keeping the backlight on and therefore it is not particularly burdensome to PTRP power.

The CMMACU 100 supports at least four sets of user interface screens. The first set of screens is the patient tracking screen, used for vital signs spot check of up to five patients at a glance and up to twenty-five patients when the patients are grouped using group identification. The second set of screens are used to associate a patient with the individual's personal data, such as name, address, social security number, contact information for next of kin. In one embodiment, the first responder/EMT can enter personal information for military personnel based on military classification (E-Enlisted, WO—Warrant Officer, O—Officer) and rank 1-9 as well as some level of unique for digit code (usually the last four digits of a Serial (Social Security) Number 0-9. The third set of screens is the first responder/EMT observations display screen, which is used to download first responder/EMT observation information to the PTRP. The fourth set of screens is used to download first responder/EMT medication selection information to the PTRP.

The following sections contain variations to the typical workflow for this embodiment of PTRP 1, CMMACU 100 and PC use cases. This list is by no means exhaustive, but certainly representative of the types of situations the CMMA may encounter during operation.

### PTRP Based Use Cases

Ops Check;
   Pass;
   Fail;
   Open package during Ops Check;
patient outside of operational temperature range;
Primary Attachment sites to patient not accessible;
   CO₂;
      No access to nose;
      Cannot dress nasal cannula behind patient's ears;
   SpO₂;
      No access to ears;
      Sensor will not adhere to ear;
   PTRP;
      Chest wound (PTRP cannot sit on chest);
      Neck wound (Cables cannot nm from head to chest);
PTRP Doesn't Report Valid Physiological Data;
   Reporting CO₂ Error;
      Kinked Hose;
      Clogged Hose;
         Excretions;
         Blood;
         Frozen H₂O;
      No gas exchange through nose;
         Patient intubated;
         Nose clogged;
      Hose not attached to patient;
         Hose falls off patent;
         Hose detaches (rips off) from PTRP;
   Reporting SpO₂ Error;
      Smashed (faulty) sensor;
      Broken/intermittent cable;
      Sensor not attached to patient;
         Sensor falls off patient;
         Sensor detaches (rips off) from PTRP;
      Inadequate Hemodynamic through attachment site;
         Cold extremities;
         Inadequate blood volume (shock);
         Tourniquet proximal to SpO₂ Attachment Site;
First responder/EMT re-attaches sensors (e.g. gap in the data);
Silent mode;
   Enter silent mode;
   Exit silent mode;
Change in patient trend indicator status (Red [symbol] Yellow [symbol] Green);
   Notification to user via PTRP;
      LED's;
      LCD;
   Data logging;
   Notification to first responder/EMT via CMMACU;
      Visual;
      Audio;
      Tactile;
Change in PTRP Status (Fully Functional [symbol] Single Fault [symbol] Multiple Fault);
On-Board (e.g. Blast & Morphine) Observation/Treatment Change;
   Error;
      Unintended selection;
      Change in assessment and/or treatment plan;
   Latent entry (documenting events when conditions permit);
PTRP hooked up to USB (e.g. charging) while hooked up to patient;
   Power Only (e.g. accessory plug);
   PC (e.g. instantiation on USB);
      Download data to PC but continue to generate log entries;
PTRP battery “died” then hooked up to USB;
   PTRP microprocessor playing possum but keeping track of time that has passed;
   PTRP battery hyper-drained, microprocessor lost power;
      PTRP battery died while packaged;
      PTRP battery died after un-packaging;
         Valid patient data previously logged into PTRP;
         PTRP not previously hooked up to patient;
Stuck Power-On button upon Removal from Package;
Shorted USB connector;
Multiple sequential PTRPs from a single patient (PC data synchronization challenge);
   Equipment failure (e.g. CO₂ clog);
   Battery “dies” (e.g. long transport issue).

CMMACU Based Use Cases
Sort patients on the screen while connected to multiple patients;
Multiple CMMACUs joining and exiting radio vicinity;
   Within range of some but not all PTRPs in the triage area;
   Out of range during patient trend indicator status transition;
      Error condition (e.g. sensor off);
      Triage level;
Multiple CMMACU's updating treatments and/or observations;
   Update all attached CMMACU(s);
   Update data log;
Enter patient personal identification information.

PC Based Use Cases
Multiple data downloads;
   Same PTRP multiple times during the same session;
   Concatenation of multiple PTRPs;
   Overlapping data from two different patients;
   Overlapping data from the same patient (e.g. 2^{nd} PTRP opened while 1^{st} still operational);
Multiple PC downloads with continuous vital signs capture.

It will be understood that various modifications and changes may be made in the present invention by those of ordinary skill in the art who have the benefit of this disclosure. All such changes and modifications fall within the spirit of this invention, the scope of which is measured by the following appended claims.

We claim:

1. A corpsman/medic medical assistant system comprising:
(i) a corpsman/medic medical assistant portable transmit/receive pack comprising a sealed housing that includes a first physiological sensor subsystem, a second physiological sensor subsystem, at least one processor running at least one algorithm, a power supply, a wireless transceiver, and memory, the sealed housing having a front panel that includes a display, at least one event keys, a treatment button for recording treatment data for treatments administered to a patient by a user, a timer, an observation button for recording observation data by the user, and a plurality of physiological trend indicators;
(ii) sensor leads for the first physiological sensor subsystem and the second physiological sensor subsystem;
wherein the display displays a plurality of physiological measurements for the patient when the sensor leads for the first physiological sensor subsystem and the second physiological sensor subsystem are connected between the patient and the transmit/receive pack, and the algorithm determines a current physiological trend status of the patient based on the plurality of physiological measurements from the first physiological sensor subsystem and the second physiological sensor subsystem and displays the current physiological trend status of the patient by activating one of the plurality of physiological trend indicators on the front panel; and
(iii) a corpsman/medic medical assistant control unit comprising an wireless transceiver,
wherein the transmit/receive pack is linked to the corpsman/medic medical assistant control unit by the wireless transceiver and the corpsman/medic medical assistant control unit receives the current physiological trend status of the patient from the transmit/receive pack.

2. The corpsman/medic medical assistant system of claim 1, wherein the corpsman/medic medical assistant control unit transmits treatment data and observation data to the transmit/receive pack and the transmit/receive pack time stamps and stores the received treatment data and observation data in memory.

3. The corpsman/medic medical assistant system of claim 1, wherein the timer is an elapsed time counter counting time from the initialization of the transmit/receive pack.

4. The corpsman/medic medical assistant system of claim 3, wherein recorded time from the timer is converted to real-time when the transmit/receive pack is connected to an external PC.

5. The corpsman/medic medical assistant system of claim 1, wherein the timer is a real-time clock that counts real-time from the initialization of the transmit/receive pack.

6. The corpsman/medic medical assistant system of claim 1, wherein the corpsman/medic medical assistant control unit is a smart phone, PDA, tablet PC or other wireless device capable of connecting and displaying vital sign measurements from at least the first physiological sensor subsystem and the second physiological sensor subsystem.

7. The corpsman/medic medical assistant system of claim 1, wherein the transmit/receive pack time stamps the plurality of physiological measurements for the patient using the timer, timestamps treatment data entered via the treatment button and observation data entered via the observation button on the front panel, and stores the plurality of physiological measurements for the patient, the treatment data and the observation data in memory.

8. The corpsman/medic medical assistant system of claim 1, wherein the corpsman/medic medical assistant control unit displays current physiological trend status for a plurality of patients on one screen.

9. The corpsman/medic medical assistant system of claim 1, wherein the algorithm compares at least one of the current physiological measurements and historical trend data of the patient with threshold physiological values for each of the plurality of physiological measurements to determine the current physiological status of the patient.

10. The corpsman/medic medical assistant system of claim 1, further comprising at least one external interface connector, wherein another external physiological sensor is connected to the transmit/receive pack via the external interface connector and the transmit/receive pack stores data received from the another external physiological sensor in memory.

11. The corpsman/medic medical assistant system of claim 10, wherein the algorithm uses the data from the at least another external physiological sensor when determining the current physiological status of the patient.

12. The corpsman/medic medical assistant system of claim 1, wherein the portable transmit/receive pack continuously determines the status of the patient when a fault condition exists for one of first physiological sensor subsystem and the second physiological sensor subsystem.

13. The corpsman/medic medical assistant system of claim 1, wherein the transmit/receive pack timestamps and records all faults from the first and second physiological sensor subsystems in memory.

14. The corpsman/medic medical assistant system of claim 1, wherein when the transmit/receive pack is connected to an external PC, the transmit/receive pack automatically formats the time stamped data recorded in memory including the physiological measurements of the patient over time, the treatment data, the observation data and sensor fault data into a spreadsheet format and a graphical format for printing.

15. The corpsman/medic medical assistant system of claim 1, wherein the transmit/receive pack records a continuous waveform for at least one of the first physiological sensor subsystem and the second physiological sensor subsystem.

16. The corpsman/medic medical assistant system of claim 1, wherein the wireless transceiver is a Bluetooth transceiver.

17. The corpsman/medic medical assistant system of claim 1, wherein the transmit/receive pack further comprises a pneumatic exhaust vent having exhaust vent fins that protect against clogging of the pneumatic exhaust vent.

18. The corpsman/medic medical assistant system of claim 1, wherein when the current physiological status of the patient changes, the transmit/receive pack displays the new physiological status of the patient by activating another of the plurality of physiological trend indicators in a blinking pattern while the one physiological trend indicator is still activated in a solid pattern for a predefined period of time.

19. The corpsman/medic medical assistant system of claim 1, wherein the transmit/receive pack is at least partially powered by an external power source through the at least one external connector.

20. The corpsman/medic medical assistant system of claim 1, wherein one of the transmit/receive packs is powered by another transmit/receive pack to retrieve and store data recorded on said one of the transmit/receive packs.

21. The corpsman/medic medical assistant system of claim 1, wherein the transmit/receive pack further comprises an operational mode switch, wherein the display, treatment and observation buttons and plurality of physiological trend indicators on the front panel are turned off by positioning the switch to a “Silent” mode.

22. The corpsman/medic medical assistant system of claim 1, further comprising means for attaching the transmit/receive pack to the patient.

23. A corpsman/medic medical assistant portable transmit receive pack comprising:
(i) a sealed housing that includes a first physiological sensor subsystem, a second physiological sensor subsystem, at least one processor running at least one algorithm, a power supply, and memory, the sealed housing having a front panel that includes a display, at least one event key, a treatment button for recording treatment data for treatments administered to a patient by a user, a timer, an observation button for recording observation data by the user and a plurality of physiological trend indicators; and
(ii) sensor leads for the first physiological sensor subsystem and at least the second physiological sensor subsystem;
wherein the display displays a plurality of physiological measurements for the patient when the sensor leads for the first physiological sensor subsystem and the second physiological sensor subsystem are connected between the patient and the transmit/receive pack, and the algorithm determines a current physiological trend status of the patient based on the plurality of physiological measurements from the first physiological sensor subsystem and the second physiological sensor subsystem and displays the current physiological trend status of the patient by activating one of the plurality of physiological trend indicators on the front panel.

24. The transmit/receive pack of claim 23, further comprising a wireless transceiver, wherein the transmit/receive pack is linked to a corpsman/medic medical assistant control unit by the wireless transceiver, the wireless transceiver transmits the current physiological status of the patient to the corpsman/medic medical assistant control unit and the corpsman/medic medical assistant control unit displays the current physiological status of the patient to the user.

25. The transmit/receive pack of claim 23, wherein the transmit/receive pack time stamps the plurality of physiological measurements for the patient using the timer and stores the plurality of physiological measurements for the patient in memory.

26. The transmit/receive pack of claim 23, wherein the transmit/receive pack timestamps treatment data entered via the treatment button and observation data entered via the observation button on the front panel of the transmit/receive pack and stores the treatment data and observation data in memory.

27. The transmit/receive pack of claim 23, wherein the transmit/receive pack receives treatment data and observation data from the corpsman/medic medical assistant control unit and the transmit/receive pack time stamps and stores the received treatment data and observation data in memory.

28. The transmit/receive pack of claim 23, wherein the algorithm compares the current physiological measurements of the patient with predetermined physiological values for each of the plurality of physiological measurements to determine the current physiological status of the patient and displays the current physiological status of the patient by activating one of the plurality of physiological trend indicators.

29. The transmit/receive pack of claim 23, further comprising at least one external interface connector, wherein another external physiological sensor is connected to the transmit/receive pack via the at least one external interface connector and the transmit/receive pack stores data received from the another external physiological sensor in memory.

30. The transmit/receive pack of claim 29, wherein the algorithm uses the data from the another external physiological sensor when determining the current physiological status of the patient.

31. The transmit/receive pack of claim 23, wherein the transmit/receive pack timestamps and records all faults from the first physiological sensor subsystem and the second physiological sensor subsystem in memory.

32. The transmit/receive pack of claim 23, wherein when the transmit/receive pack is connected to an external PC, the transmit/receive pack automatically formats the time stamped data recorded in memory including the physiological measurements of the patient over time, treatment data, observation data and sensor fault data into a spreadsheet format and a graphical format for printing.

33. The transmit/receive pack of claim 23, wherein the timing source is an elapsed time counter and the elapsed time counter counts time from the initialization of the transmit/receive pack.

34. The transmit/receive pack of claim 33, wherein the recorded time from the timer is converted to real-time when the transmit/receive pack is connected to an external PC.

35. The transmit/receive pack of claim 23, wherein the timer is a real-time clock that counts real-time from the initialization of the transmit/receive pack.

36. The transmit/receive pack of claim 23, wherein the transmit/receive pack records a continuous waveform for at least one of the first physiological sensor subsystem and the second physiological sensor subsystem

37. The transmit/receive pack of claim 23, wherein the wireless transceiver is a Bluetooth transceiver.

38. The transmit/receive pack of claim 23, further comprising at least one of a temperature sensor, a humidity sensor and an atmospheric pressure sensor, wherein the transmit/receive pack timestamps and records data received from said at least one sensor in memory.

39. The transmit/receive pack of claim 23, further comprising a pneumatic exhaust vent having exhaust vent fins that protect against clogging of the pneumatic exhaust vent.

40. The transmit/receive pack of claim 28, wherein when the current physiological status of the patient changes, the transmit/receive pack displays the new physiological status of the patient by activating another of the plurality of physiological trend indicators in a blinking pattern while the one physiological trend indicator is still activated in a solid pattern for a predefined period of time.

41. The transmit/receive pack of claim 23, wherein the power supply is a rechargeable battery.

42. The transmit/receive pack of claim 23, wherein the transmit/receive pack is powered at least partially by an external power source through the at least one external connector.

43. The transmit/receive pack of claim 23, wherein a first transmit/receive pack is powered by a second transmit/receive pack to retrieve and store data recorded on the first transmit/receive pack.

44. The transmit/receive pack of claim 23, wherein the transmit/receive pack retains sequential time in memory for at least 72 hours after the power source is drained and does not support operation of the transmit/receive pack.

45. The transmit/receive pack of claim 23, wherein the transmit/receive pack retains data stored in memory for at least 30 days after the power source is drained and does not support operation of the transmit/receive pack.

46. The transmit/receive pack of claim 23, further comprising an operational mode switch, wherein the display, treatment and observation buttons and plurality of physiological trend indicators on the front panel of the transmit/receive pack are turned off by activating the operational mode switch to the “Silent” mode.

47. The transmit/receive pack of claim 23, further comprising a sealed package enclosing the transmit/receive pack, wherein the transmit/receive pack automatically initializes when removed from the sealed package.

48. The transmit/receive pack of claim 23, wherein the transmit/receive pack performs an operational system status check in the sealed package without compromising the sealed package.

49. The transmit/receive pack of claim 23, further comprising means for attaching the transmit/receive pack to the patient.

50. The transmit/receive pack of claim 28, wherein a red physiological trend indicator is physically offset from a green physiological trend indicator and a yellow physiological trend indicator.
